(19) United States
(12) Patent Application Publication
(10) Pub. No.: US 2011/0135611 A1
(43) Pub. Date: Jun. 9, 2011

(54) METHODS FOR TREATING APOLIPOPROTEIN E4-ASSOCIATED DISORDERS

(75) Inventors: Yadong HUANG, San Francisco, CA (US); Gang Li, Albany, CA (US)

(73) Assignee: The J. David Gladstone Institutes, San Francisco, CA (US)

(21) Appl. No.: 12/958,052

(22) Filed: Dec. 1, 2010

Related U.S. Application Data
(60) Provisional application No. 61/266,414, filed on Dec. 3, 2009.

(51) Int. Cl. A61K 35/12; A61P 25/00

(52) U.S. Cl. 424/93.7

(57) ABSTRACT

The present disclosure provides methods of reducing apoE4 fragment-mediated toxicity of interneurons, e.g, GABAergic interneurons. The present disclosure provides methods of treating apoE4-mediated neurological disorders in an apoE4-positive individual.

### CROSS-REFERENCE

This application claims the benefit of U.S. Provisional Patent Application No. 61/266,414, filed Dec. 3, 2009, which application is incorporated herein by reference in its entirety.

### BACKGROUND

Apolipoprotein (apo) E, a polymorphic protein with three isoforms (apoE2, apoE3, and apoE4), is essential for lipid homeostasis. Carriers of apoE4 are at higher risk for developing Alzheimer's Disease (AD). The hippocampus is one of the first regions of the brain damaged in AD, and memory deficits and disorientation are among the early symptoms.

In the mammalian central nervous system, new neurons are generated throughout life. In adults, active neurogenesis occurs in two brain regions. One is the subgranular zone (SGZ) of the dentate gyrus in the hippocampus where newly generated neurons may participate in learning and memory formation. The other is the subventricular zone (SVZ) of the lateral ventricle, where new neurons migrate to the olfactory bulb. Generally, adult neurogenesis proceeds through four developmental stages: (1) proliferation of neural stem/progenitor cells (NSCs), (2) neuronal fate determination of NSCs, (3) maturation and migration of new neurons, and (4) functional integration of new neurons into existing neuronal circuits. Adult neurogenesis is regulated by several factors, including transcription factors, hormones, neurotransmitters, cell niches, exercise, and specific molecules.

### LITERATURE

Marcade et al. (2008) J. Neurochem. 106:392; Mohr et al. (1986) Clin. Neuropharmacol. 9:257; Lanctôt et al. (2004)Can. J. Psychiatry 49:439; Lauzada et al. (2004) FASEB J. 18:511; U.S. Patent Publication No. 2007/0112017.

### SUMMARY

The present disclosure provides methods of reducing apoE4 fragment-mediated toxicity of interneurons, e.g, GABAergic interneurons. The present disclosure provides methods of treating apoE4-mediated neurological disorders in an apoE4-positive individual.

### BRIEF DESCRIPTION OF THE DRAWINGS

FIGS. 1A-J depict expression of apoE in hippocampal neural stem cells.

FIGS. 2A-P depict hippocampal neurogenesis and astrogenesis in mice with knockout (KO) for apoE or with knockin (KI) alleles for human apoE3 or apoE4.

FIGS. 3A-K depict dendritic development of newborn neurons in the hippocampus in apoE4-KI mice.

FIGS. 4A-H depict numbers of GABAergic interneurons and GABA release in the hippocampus of apoE4-KI mice.

FIGS. 5A-O depict levels of neurotoxic apoE fragments, tau phosphorylation, and GABAergic neuron survival in primary hippocampal neuronal cultures from apoE4-KI mice.

FIGS. 6A-G depict GABAergic electrophysiological inputs to newborn neurons in the hippocampus of apoE4-KI mice.

FIGS. 7A-E depict the effect of GABA_{A} receptor potentiator on hippocampal neurogenesis in apoE4-KI mice.

### DEFINITIONS

As used herein, “GABA_{A} receptor” refers to a heteropentameric ligand-gated ion channel. Binding of gamma-aminobutyric acid (GABA) to the GABA_{A} receptor increases the permeability of neuronal membranes to chloride ions. Native GABA_{A} receptors are formed from at least 19 related subunits. The subunits are grouped into alpha, beta, delta, epsilon, pi, and rho families. The most prevalent combinations of GABA_{A} receptors are: a stoichiometric combination of the 2 alpha, 2 beta, and 1 gamma subunits; and a stoichiometric combination of the 2 alpha, 2 beta, and 1 delta subunits. The adult brain predominantly expresses the alpha₁ beta₂ gamma₂ subunit combination (60%) with the alpha₂ beta₃ gamma₂ and alpha₃ betaₙ gamma₂ subunits comprising the majority (35%) of the remaining receptors. The relative effects of GABA are influenced by the GABA_{A} receptor subunit expressed in a specific brain region or neuronal circuit. All known GABA_{A} receptors contain a plurality of distinct modulatory sites, which can include the benzodiazepine (BZ) binding site; and allosteric sites for picrotoxin, barbiturates, neuroactive steroids, and ethanol.

As used herein, an “apoE4-associated disorder” is any disorder that is caused by the presence of apoE4 in a cell, in the serum, in the interstitial fluid, in the cerebrospinal fluid, or in any other bodily fluid of an individual; any physiological process or metabolic event that is influenced by apoE4 domain interaction; any disorder that is characterized by the presence of apoE4; a symptom of a disorder that is caused by the presence of apoE4 in a cell or in a bodily fluid; a phenomenon associated with a disorder caused by the presence in a cell or in a bodily fluid of apoE4; and the sequelae of any disorder that is caused by the presence of apoE4. ApoE4-associated disorders include apoE4-associated neurological disorders and disorders related to high serum lipid levels. ApoE4-associated neurological disorders include, but are not limited to, sporadic Alzheimer's disease; familial Alzheimer's disease; poor outcome following a stroke; poor outcome following traumatic head injury; and cerebral ischemia. Phenomena associated with apoE4-associated neurological disorders include, but are not limited to, neurofibrillary tangles; amyloid deposits; memory loss; and a reduction in cognitive function. ApoE4-related disorders associated with high serum lipid levels include, but are not limited to, atherosclerosis, and coronary artery disease. Phenomena associated with such apoE4-associated disorders include high serum cholesterol levels.

The term “Alzheimer's disease” (abbreviated herein as “AD”) as used herein refers to a condition associated with formation of neuritic plaques comprising amyloid β protein primarily in the hippocampus and cerebral cortex, as well as impairment in both learning and memory. “AD” as used herein is meant to encompass both AD as well as AD-type pathologies.

The term “phenomenon associated with Alzheimer's disease” as used herein refers to a structural, molecular, or functional event associated with AD, particularly such an event that is readily assessable in an animal model. Such events include, but are not limited to, amyloid deposition, neuropathological developments, learning and memory deficits, and other AD-associated characteristics.

As used herein, the term “neural stem cell” (NSC) refers to an undifferentiated neural cell that can proliferate, self-renew, and differentiate into the main adult neural cells of the brain. NSCs are capable of self-maintenance (self-renewal), meaning that with each cell division, one daughter cell will also be a stem cell. The non-stem cell progeny of NSCs are termed neural progenitor cells. Neural progenitors cells generated from a single multipotent NSC are capable of differentiating into neurons, astrocytes (type I and type II), and oligodendrocytes. Hence, NSCs are “multipotent” because their progeny have multiple neural cell fates. Thus, NSCs can be functionally defined as a cell with the ability to: 1) proliferate, 2) self-renew, and 3) produce functional progeny that can differentiate into the three main cell types found in the central nervous system: neurons, astrocytes and oligodendrocytes.

As used herein, the terms “neural progenitor cell” or “neural precursor cell” refer to a cell that can generate progeny such as neuronal cells (e.g., neuronal precursors or mature neurons), glial precursors, mature astrocytes, or mature oligodendrocytes. Typically, the cells express some of the phenotypic markers that are characteristic of the neural lineage. A “neuronal progenitor cell” or “neuronal precursor cell” is a cell that can generate progeny that are mature neurons. These cells may or may not also have the capability to generate glial cells.

A “neurosphere” is a group of cells derived from a single neural stem cell as the result of clonal expansion. A method for culturing neural stem cells to form neurospheres has been described in, for example, U.S. Pat. No. 5,750,376.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc.

A “therapeutically effective amount” or “efficacious amount” refers to the amount of a compound or a number of cells that, when administered to a mammal or other subject for treating a disease, is sufficient to effect such treatment for the disease. The “therapeutically effective amount” will vary depending on the compound or the cell, the disease and its severity and the age, weight, etc., of the subject to be treated.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a GABA_{A} receptor agonist” includes a plurality of such agonists and reference to “the neural stem cell” includes reference to one or more neural stem cells and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods of reducing neurotoxic apoE4 fragment-mediated interneuron dysfunction, e.g., GABAergic interneuron dysfunction. Reducing apoE4 neurotoxic fragment-mediated interneuron dysfunction results in an increase in the number of functional GABAergic interneurons. Increasing the number of functional GABAergic interneurons, e.g., in the hilus of the dentate gyrus, can increase adult neurogenesis and can treat apoE4-mediated neurological disorders. The present disclosure thus provides methods of treating apoE4-mediated neurological disorders in an apoE4-positive individual.

A subject method generally involves enhancing GABAergic function in an individual in need thereof. Enhancing GABAergic function can be achieved by one or more of: 1) stimulating or enhancing GABA release from a GABAergic interneuron; 2) inhibiting the breakdown of GABA; 3) contacting a GABA_{A} receptor (e.g., in a GABAergic interneuron with a GABA_{A} receptor agonist; and 4) selectively inhibiting GABA reuptake; etc.

Thus, in some embodiments, a subject method generally involves administering to an individual in need thereof (e.g., an individual having an apoE4-mediated neurological disorder) an effective amount of one or more of: 1) an agent that stimulates or enhances release of GABA, e.g., from a GABAergic interneuron; 2) an agent that inhibits GABA-transaminase; 3) a gamma amino butyric acid-A (GABA_{A}) receptor agonist; and 4) a selective GABA reuptake inhibitor. A GABA_{A} receptor agonist (or an agent that stimulates or enhances release of GABA, e.g., from a GABAergic interneuron; or an agent that inhibits GABA-transaminase; or a selective GABA reuptake inhibitor) can be administered alone (e.g., as monotherapy), in conjunction with another therapeutic agent (e.g., combination therapy), or in conjunction with a stem cell therapy. For example, the present disclosure provides a method of treating an apoE4-associated disorder in an individual, the method generally involving: a) administering to the individual an effective amount of a GABA_{A} receptor agonist; and b) introducing exogenous NSCs into the individual. A GABA_{A} receptor agonist, when administered in conjunction with stem cell therapy, can increase the survival time of the introduced NSCs and/or increasing neuronal differentiation of the introduced NSCs.

Native GABA_{A} receptors are formed from at least 19 related subunits. The subunits are grouped into alpha, beta, delta, epsilon, pi, and rho families. The most prevalent combinations of GABA_{A} receptors are: a stoichiometric combination of the 2 alpha, 2 beta, and 1 gamma subunits; and a stoichiometric combination of the 2 alpha, 2 beta, and 1 delta subunits. The adult brain predominantly expresses the alpha₁ beta₂ gamma₂ subunit combination (60%) with the alpha₂ beta₃ gamma₂ and alpha₃ betaₙ gamma₂ subunits comprising the majority (35%) of the remaining receptors. Amino acid sequences of GABA_{A} receptor subunits are known. The following are merely exemplary sources of amino acid sequences of the various subunits: GenBank Accession No. NP_{—}002033.2: rho1; GenBank Accession No. NP_{—}002034.2: rho2; GenBank Accession No. CAA70904.1: epsilon; GenBank Accession No. CAA01921.1: alpha-6; GenBank Accession No. CAA01920.1: alpha-5; GenBank Accession No. NP_{—}000800.2: alpha-4; GenBank Accession No. AAG12455.1: alpha-3; GenBank Accession No. AAB27278.1: alpha-2; GenBank Accession No. NP_{—}001121120.1; PCT Publication No. WO 92/22562: various alpha subunits; NP_{—}000803.2: beta-1; GenBank Accession No. AAB33983.1: beta-2; GenBank Accession No. NP_{—}000805.1: beta-3; GenBank Accession No. NP_{—}000806.2: delta; GenBank Accession No. NP_{—}944494.1: gamma-2; and GenBank Accession No. NP_{—}775807.2: gamma-1. See also: Burt and Kamatchi (1991) FASEB J. 5:2916; Mitchell et al. (2008) Neurochem. Int. 52:588; Davies et al. (1996)Front. Biosci. 1:d214; Böhme et al. (2004) J. Biol. Chem. 279:35193; Schonfield et al. (1989) FEBS Lett. 244:361; and Jin et al. (2004) J. Biol. Chem. 279:14179.

In some embodiments, a subject method is effective to increase the number of newborn mature neurons in the SGZ of the hippocampus of an individual by at least about 5%, at least about 10%, at least about 15%, at least about 20%, at least about 25%, at least about 30%, at least about 40%, at least about 50%, or more than 50%, compared with the number of newborn mature neurons in the SGZ of the hippocampus in the absence of treatment, or before treatment, with the method. Thus, e.g., in some embodiments, an effective amount of a GABA_{A} receptor agonist (or an agent that stimulates or enhances release of GABA, e.g., from a GABAergic interneuron; or an agent that inhibits GABA-transaminase; or a selective GABA reuptake inhibitor) is an amount that is effective, when administered in one or more doses, in monotherapy or in combination therapy (e.g., in combination with stem cell therapy or in combination therapy with at least one additional therapeutic agent), to increase the number of newborn mature neurons in the SGZ of the hippocampus by at least about 5%, at least about 10%, at least about 15%, at least about 20%, at least about 25%, at least about 30%, at least about 40%, at least about 50%, or more than 50%, compared with the number of newborn mature neurons in the SGZ of the hippocampus in the absence of treatment, or before treatment, with the GABA_{A} receptor agonist.

In some embodiments, a subject method is effective to increase the number of GAD67-positive GABAergic interneurons in the hilus of the hippocampus of an individual by at least about 5%, at least about 10%, at least about 15%, at least about 20%, at least about 25%, at least about 30%, at least about 40%, at least about 50%, or more than 50%, compared with the number of GAD67-positive GABAergic interneurons in the hilus of the hippocampus of the individual in the absence of treatment, or before treatment, with the method. Thus, e.g., in some embodiments, an effective amount of a GABA_{A} receptor agonist (or an agent that stimulates or enhances release of GABA, e.g., from a GABAergic interneuron; or an agent that inhibits GABA-transaminase; or a selective GABA reuptake inhibitor) is an amount that is effective, when administered in one or more doses, in monotherapy or in combination therapy (e.g., in combination with stem cell therapy or in combination therapy with at least one additional therapeutic agent), to increase the number of GAD67-positive GABAergic interneurons in the hilus of the hippocampus of an individual by at least about 5%, at least about 10%, at least about 15%, at least about 20%, at least about 25%, at least about 30%, at least about 40%, at least about 50%, or more than 50%, compared with the number of GAD67-positive GABAergic interneurons in the hilus of the hippocampus of the individual in the absence of treatment, or before treatment, with the GABA_{A} receptor agonist.

In some embodiments, a subject method is effective to increase the functionality of a GABAergic interneuron in the hippocampus of an individual by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared with the functionality of the GABAergic interneuron in the hippocampus of the individual in the absence of treatment, or before treatment, with the subject method. For example, in some embodiments, an effective amount of a GABA_{A} receptor agonist (or an agent that stimulates or enhances release of GABA, e.g., from a GABAergic interneuron; or an agent that inhibits GABA-transaminase; or a selective GABA reuptake inhibitor) is an amount that is effective, when administered in one or more doses, in monotherapy or in combination therapy (e.g., in combination with stem cell therapy or in combination therapy with at least one additional therapeutic agent), to increase the functionality of a GABAergic interneuron in the hippocampus of an individual by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared with the functionality of the GABAergic interneuron in the hippocampus of the individual in the absence of treatment, or before treatment, with the GABA_{A} receptor agonist.

The functionality of a GABAergic interneuron includes basal GABA release, KCl-evoked GABA release, and neuregulin-evoked GABA release. Thus, e.g., in some embodiments, an effective amount of a GABA_{A} receptor agonist is an amount that is effective, when administered in one or more doses, in monotherapy or in combination therapy (e.g., in combination with stem cell therapy or in combination therapy with at least one additional therapeutic agent), to increase the amount of GABA released by GABAergic interneurons in the hippocampus of an individual by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared with the amount of GABA released by GABAergic interneurons in the hippocampus of the individual in the absence of treatment, or before treatment, with the GABA_{A} receptor agonist.

In some embodiments, a subject method is effective to ameliorate at least one phenomenon associated with an apoE4-associated neurological disorder, where such phenomena include, e.g., neurofibrillary tangles; amyloid deposits; memory loss; and a reduction in cognitive function. Thus, for example, in some embodiments, a subject method is effective to reduce memory loss and at least slow the reduction in cognitive function. For example, in some embodiments, a subject method is effective to increase memory function and/or to increase cognitive function. Thus, e.g., in some embodiments, an effective amount of a GABA_{A} receptor agonist (or an agent that stimulates or enhances release of GABA, e.g., from a GABAergic interneuron; or an agent that inhibits GABA-transaminase; or a selective GABA reuptake inhibitor) is an amount that is effective, when administered in one or more doses, in monotherapy or in combination therapy (e.g., in combination with stem cell therapy or in combination therapy with at least one additional therapeutic agent), to reduce memory loss, to increase memory functions, to reduce loss of cognitive function, or to increase cognitive function.

### Stem Cell Therapy

As noted above, in some embodiments, a GABA_{A} receptor agonist (or an agent that stimulates or enhances release of GABA, e.g., from a GABAergic interneuron; or an agent that inhibits GABA-transaminase; or a selective GABA reuptake inhibitor) is administered in conjunction with stem cell therapy. For example, in some embodiments, a subject method involves: a) administering to an individual in need thereof an effective amount of a GABA_{A} receptor agonist; and b) introducing a stem cell into the individual. As another example, in some embodiments, a subject method involves: a) administering to an individual in need thereof an effective amount of a GABA_{A} receptor agonist; and b) introducing an induced pluripotent stem (iPS) cell into the individual. As another example, in some embodiments, a subject method involves: a) administering to an individual in need thereof an effective amount of a GABA_{A} receptor agonist; and b) introducing a neural stem cell (NSC) into the individual. As another example, in some embodiments, a subject method involves: a) administering to an individual in need thereof an effective amount of a GABA_{A} receptor agonist; and b) introducing an induced neural stem cell (iNSC) into the individual.

A GABA_{A} receptor agonist (or other agent, as noted above) is generally administered to an individual before and/or concurrently with, introduction of stem cells into the individual. For example, a GABA_{A} receptor agonist (or an agent that stimulates or enhances release of GABA, e.g., from a GABAergic interneuron; or an agent that inhibits GABA-transaminase; or a selective GABA reuptake inhibitor) can be administered to an individual from 15 minutes to about 30 minutes, from about 30 minutes to about 60 minutes, from about 60 minutes to about 2 hours, from about 2 hours to about 4 hours, from about 4 hours to about 8 hours, from about 8 hours to about 16 hours, from about 16 hours to about 24 hours, from about 1 day to about 2 days, from about 2 days to about 4 days, from about 4 days to about 7 days, from about 1 week to about 2 weeks, or from about 2 weeks to about 4 weeks, in advance of introduction of a stem cell into the individual.

The stem cells used for transplantation can be allogeneic, autologous, or xenogeneic, relative to the individual being treated (e.g., the individual into whom the stem cells are being transplanted). For example, in some cases, the stem cells (e.g., NSC or iNSC) are obtained from a human donor individual who is the same as the human individual being treated (the recipient). As another example, in some cases the stem cells (e.g., NSC or iNSC) are obtained from a human donor individual who is other than the human individual being treated (the recipient).

Neural stem cells of various species have been described. See, e.g., WO 93/01275, WO 94/09119, WO 94/10292, WO 94/16718, and Cattaneo et al., Mol. Brain. Res., 42, pp. 161-66 (1996). In some embodiments, NSCs, when maintained in certain culture conditions (e.g., a mitogen-containing (e.g., epidermal growth factor or epidermal growth factor plus basic fibroblast growth factor), serum-free culture medium), grow in suspension culture to form aggregates of cells known as “neuro spheres.”

NSCs can be generated from somatic cells (where the NSCs are referred to as “induced NSCs”); pluripotent stem cells; induced pluripotent stem cells (iPS); or fetal or adult tissue that contains NSCs. Suitable tissue sources of NSCs include, but are not limited to, hippocampus, septal nuclei, cortex, cerebellum, ventral mesencephalon, and spinal cord.

A suitable NSC exhibits one or more of the following properties: 1) expression of Nestin; 2) expression of Sox2; 3) expression of Musashi1; 4) ability to undergo self-renewal, either as a monolayer or in suspension cultures as neurospheres; 5) ability to differentiate into neurons, specific subtypes of neurons, astrocytes, and oligodendrocytes; and 6) morphological characteristics typical for NSCs. A suitable iNSC can also express CD133 and Vimentin. Nestin, Sox2, and Musashi1 are well described in the literature as hallmark genes expressed in NSCs. See, e.g., GenBank Accession Nos. NP_{—}006608, CAA46780, and CAI16338 for Nestin. For Musashi1, see, e.g., GenBank Accession No. BAB69769; and Shu et al. (2002) Biochem. Biophys. Res. Comm. 293:150.

A suitable NSC is generally negative for markers that identify mature neurons, astrocytes, and oligodendrocytes. Thus, e.g., a suitable NSC is generally microtubule-associated protein-2 (MAP2) negative, neuron-specific nuclear protein (NeuN) negative, Tau negative, S100β negative, oligodendrocyte marker O4 negative, and oligodendrocyte lineage transcription factor Olig2 negative. These markers of mature neural markers are well described in the literature. For MAP2, see, e.g., GenBank Accession Nos. AAA59552, AAB48098, AAI43246, and AAH38857. For NeuN, see, e.g., Wolf et al. (1996) J. Histochem. & Cytochem. 44:1167. For S100β, see, e.g., GenBank Accession Nos. NP_{—}006263.1 (H. sapiens S100β); NP_{—}033141 (Mus musculus S100β (3); CAG46920.1 (Homo sapiens S100β (3); and see also, Allore et al. (1990) J. Biol. Chem. 265:15537. For O4, see, e.g., Schachner et al. (1981) Dev. Biol. 83:328; Bansal et al. (1989(J. Neurosci. Res. 24:548; and Bansal and Pfeiffer (1989) Proc. Natl. Acad. Sci. USA 86:6181. For Olig2, see, e.g., Lu et al. (2001) Proc. Natl. Acad. Sci. USA 98:10851; Ligon et al. (2004) J. Neuropathol. Exp. Neurol. 63:499.

### Tissue Sources

Suitable tissue sources of neural stem cells include the CNS, including the cerebral cortex, cerebellum, midbrain, brainstem, spinal cord and ventricular tissue; and areas of the peripheral nervous system (PNS) including the carotid body and the adrenal medulla. Exemplary areas include regions in the basal ganglia, e.g., the striatum which consists of the caudate and putamen, or various cell groups, such as the globus pallidus, the subthalamic nucleus, the nucleus basalis, or the substantia nigra pars compacta. In some embodiments, the neural tissue is obtained from ventricular tissue that is found lining CNS ventricles (e.g., lateral ventricles, third ventricle, fourth ventricle, central canal, cerebral aqueduct, etc.) and includes the subependyma.

Non-autologous human neural stem cells can be derived from fetal tissue following elective abortion, or from a post-natal, juvenile, or adult organ donor. Autologous neural tissue can be obtained by biopsy, or from patients undergoing neurosurgery in which neural tissue is removed, for example, during epilepsy surgery, temporal lobectomies and hippocampalectomies. Neural stem cells have been isolated from a variety of adult CNS ventricular regions, including the frontal lobe, conus medullaris, thoracic spinal cord, brain stem, and hypothalamus. In each of these cases, the neural stem cell exhibits self-maintenance and generates a large number of progeny which include neurons, astrocytes and oligodendrocytes.

### Induced NSCs

Suitable NSCs include induced NSCs (iNSCs). An iNSC can be generated by introducing into a somatic cell one or more of: an exogenous Sox2 polypeptide, an Oct-3/4 polypeptide, an exogenous c-Myc polypeptide, an exogenous Klf4 polypeptide, an exogenous Nanog polypeptide, and an exogenous Lin28 polypeptide.

Sox2 polypeptides, Oct-3/4 polypeptides, c-Myc polypeptides, and Klf4 polypeptides, are known in the art and are described in, e.g., U.S. Patent Publication No. 2009/0191159. Nanog polypeptides and Lin28 polypeptides are known in the art and are described in, e.g., U.S. Patent Publication No. 2009/0047263. See also the following GenBank Accession Nos.: 1) GenBank Accession Nos. NP_{—}002692, NP_{—}001108427; NP_{—}001093427; NP_{—}001009178; and NP_{—}038661 for Oct-3/4; 2) GenBank Accession Nos. NP_{—}004226, NP_{—}001017280, NP_{—}057354, AAP36222, NP_{—}034767, and NP_{—}446165 for Klf4 and Klf4 family members; 3) GenBank Accession Nos. NP_{—}002458, NP_{—}001005154, NP_{—}036735, NP_{—}034979, P0C0N9, and NP_{—}001026123 for c-Myc; 4) GenBank Accession Nos. AAP49529 and BAC76999, for Nanog; 5) GenBank Accession Nos. AAH28566 and NP_{—}078950, for Lin28; and 6) GenBank Accession Nos: NP_{—}003097, NP_{—}001098933, NP_{—}035573, ACA58281, BAA09168, NP_{—}001032751, and NP_{—}648694 for Sox2 amino acid sequences.

A multipotent iNSC can be induced from a wide variety of mammalian somatic cells. Examples of suitable mammalian cells include, but are not limited to: fibroblasts (including dermal fibroblasts, human foreskin fibroblasts, etc.), bone marrow-derived mononuclear cells, skeletal muscle cells, adipose cells, peripheral blood mononuclear cells, macrophages, hepatocytes, keratinocytes, oral keratinocytes, hair follicle dermal cells, gastric epithelial cells, lung epithelial cells, synovial cells, kidney cells, skin epithelial cells, and osteoblasts.

A somatic cell can also originate from many different types of tissue, e.g., bone marrow, skin (e.g., dermis, epidermis), muscle, adipose tissue, peripheral blood, foreskin, skeletal muscle, or smooth muscle. The cells can also be derived from neonatal tissue, including, but not limited to: umbilical cord tissues (e.g., the umbilical cord, cord blood, cord blood vessels), the amnion, the placenta, or other various neonatal tissues (e.g., bone marrow fluid, muscle, adipose tissue, peripheral blood, skin, skeletal muscle etc.

A somatic cell can be obtained from any of a variety of mammals, including, e.g., humans, non-human primates, murines (e.g., mice, rats), ungulates (e.g., bovines, equines, ovines, caprines, etc.), felines, canines, etc.

A somatic cell can be derived from neonatal or post-natal tissue collected from a subject within the period from birth, including cesarean birth, to death. For example, the tissue may be from a subject who is >10 minutes old, >1 hour old, >1 day old, >1 month old, >2 months old, >6 months old, >1 year old, >2 years old, >5 years old, >10 years old, >15 years old, >18 years old, >25 years old, >35 years old, >45 years old, >55 years old, >65 years old, >80 years old, <80 years old, <70 years old, <60 years old, <50 years old, <40 years old, <30 years old, <20 years old or <10 years old. The subject may be a neonatal infant. In some cases, the subject is a child or an adult. In some examples, the tissue is from a human of age 2, 5, 10 or 20 hours. In other examples, the tissue is from a human of age 1 month, 2 months, 3 months, 4 months, 5 months, 6 months, 9 months or 12 months. In some cases, the tissue is from a human of age 1 year, 2 years, 3 years, 4 years, 5 years, 18 years, 20 years, 21 years, 23 years, 24 years, 25 years, 28 years, 29 years, 31 years, 33 years, 34 years, 35 years, 37 years, 38 years, 40 years, 41 years, 42 years, 43 years, 44 years, 47 years, 51 years, 55 years, 61 years, 63 years, 65 years, 70 years, 77 years, or 85 years old.

The cells can be from non-embryonic tissue, e.g., at a stage of development later than the embryonic stage. In other cases, the cells may be derived from an embryo. In some cases, the cells may be from tissue at a stage of development later than the fetal stage. In other cases, the cells may be derived from a fetus.

The cells to be induced or reprogrammed can be obtained from a single cell or a population of cells. The population may be homogeneous or heterogeneous. The cells can be a population of cells found in a human cellular sample, e.g., a biopsy or blood sample.

Methods for obtaining human somatic cells are well established, as described in, e.g., Schantz and Ng (2004), A Manual for Primary Human Cell Culture, World Scientific Publishing Co., Pte, Ltd. In some cases, the methods include obtaining a cellular sample, e.g., by a biopsy (e.g., a skin sample), blood draw, or alveolar or other pulmonary lavage. It is to be understood that initial plating densities of cells prepared from a tissue can vary, due to a variety of factors, e.g., expected viability or adherence of cells from that particular tissue.

An exogenous polypeptide can be introduced into a somatic cell by contacting the somatic cell with the exogenous polypeptide (e.g., a Sox2 polypeptide, as described above) wherein the exogenous polypeptide is taken up into the cell.

In some embodiments, an exogenous polypeptide (e.g., a Sox2 polypeptide) comprises a protein transduction domain, e.g., an exogenous polypeptide is linked, covalently or non-covalently, to a protein transduction domain.

“Protein Transduction Domain” or PTD refers to a polypeptide, polynucleotide, carbohydrate, or organic or inorganic compound that facilitates traversing a lipid bilayer, micelle, cell membrane, organelle membrane, or vesicle membrane. A PTD attached to another molecule facilitates the molecule traversing a membrane, for example going from extracellular space to intracellular space, or cytosol to within an organelle. In some embodiments, a PTD is covalently linked to the amino terminus of an exogenous polypeptide (e.g., a Sox2 polypeptide). In some embodiments, a PTD is covalently linked to the carboxyl terminus of an exogenous polypeptide (e.g., a Sox2 polypeptide). Exemplary protein transduction domains include but are not limited to a minimal undecapeptide protein transduction domain (corresponding to residues 47-57 of HIV-1 TAT comprising YGRKKRRQRRR; SEQ ID NO:1); a polyarginine sequence comprising a number of arginines sufficient to direct entry into a cell (e.g., 3, 4, 5, 6, 7, 8, 9, 10, or 10-50 arginines); a VP22 domain (Zender et al., Cancer Gene Ther. 2002 June; 9(6):489-96); an Drosophila Antennapedia protein transduction domain (Noguchi et al., Diabetes 2003; 52(7):1732-1737); a truncated human calcitonin peptide (Trehin et al. Pharm. Research, 21:1248-1256, 2004); polylysine (Wender et al., PNAS, Vol. 97:13003-13008); RRQRRTSKLMKR (SEQ ID NO:2); Transportan GWTLNSAGYLLGKINLKALAALAKKIL (SEQ ID NO:3); KALAWEAKLAKALAKALAKHLAKALAKALKCEA (SEQ ID NO:4); and RQIKIWFQNRRMKWKK (SEQ ID NO:5). Exemplary PTDs include but are not limited to, YGRKKRRQRRR (SEQ ID NO:1), RKKRRQRRR (SEQ ID NO:6); an arginine homopolymer of from 3 arginine residues to 50 arginine residues; Exemplary PTD domain amino acid sequences include, but are not limited to, any of the following: YGRKKRRQRRR (SEQ ID NO:1); RKKRRQRR (SEQ ID NO:6); YARAAARQARA (SEQ ID NO:7); THRLPRRRRRR (SEQ ID NO:8); and GGRRARRRRRR (SEQ ID NO:9).

In some embodiments, introduction of an exogenous polypeptide (e.g., an exogenous Sox2 polypeptide) into a somatic cell is achieved by genetic modification of the somatic cell with an exogenous nucleic acid comprising a nucleotide sequence encoding the polypeptide. Exogenous nucleic acids include a recombinant expression vector comprising a nucleotide sequence encoding an exogenous polypeptide (e.g., an exogenous Sox2 polypeptide). Suitable recombinant expression vectors include plasmids, as well as viral-based expression vectors, e.g., lentivirus vectors, adenovirus vectors, adeno-associated virus vectors, etc., which are well known in the art.

### iPS Cells

In some embodiments, NSCs are generated from induced pluripotent stem (iPS) cells. iPS cells are generated from somatic cells, including skin fibroblasts, using, e.g., known methods. iPS cells produce and express on their cell surface one or more of the following cell surface antigens: SSEA-3, SSEA-4, TRA-1-60, TRA-1-81, TRA-2-49/6E, and Nanog. In some embodiments, iPS cells produce and express on their cell surface SSEA-3, SSEA-4, TRA-1-60, TRA-1-81, TRA-2-49/6E, and Nanog. iPS cells express one or more of the following genes: Oct-3/4, Sox2, Nanog, GDF3, REX1, FGF4, ESG1, DPPA2, DPPA4, and hTERT. In some embodiments, an iPS cell expresses Oct-3/4, Sox2, Nanog, GDF3, REX1, FGF4, ESG1, DPPA2, DPPA4, and hTERT. iPS can be induced to differentiate into neural cells that express one or more of: βIII-tubulin, tyrosine hydroxylase, AADC, DAT, ChAT, LMX1B, and MAP2. Methods of generating iPS are known in the art, and any such method can be used to generate iPS. See, e.g., Takahashi and Yamanaka (2006) Cell 126:663-676; Yamanaka et. al. (2007) Nature 448:313-7; Wernig et. al. (2007) Nature 448:318-24; Maherali (2007) Cell Stem Cell 1:55-70.

iPS cells can be generated from somatic cells (e.g., skin fibroblasts) by genetically modifying the somatic cells with one or more expression constructs encoding Oct-3/4 and Sox2. In some embodiments, somatic cells are genetically modified with one or more expression constructs comprising nucleotide sequences encoding Oct-3/4, Sox2, c-myc, and Klf4. In some embodiments, somatic cells are genetically modified with one or more expression constructs comprising nucleotide sequences encoding Oct-4, Sox2, Nanog, and LIN28.

iPS cells can be induced to differentiate into neural cells using any of a variety of published protocols (see, e.g., Muotri et al., 2005, Proc. Natl. Acad. Sci. USA. 102:18644; Takahashi et al, 2007, Cell 131:861). For example, in some embodiments, iPS cells are cultured on mitotically inactivated (e.g., mitomycin C-treated or irradiated) mouse embryonic fibroblasts (Specialty Media, Lavellette, N.J.) in DMEM/F12 Glutamax (GIBCO), 20% knockout serum replacement (GIBCO), 0.1 mM nonessential amino acids (GIBCO), 0.1 mM 2-mercaptoethanol (GIBCO), and 4 ng/ml bFGF-2 (R &. D Systems). iPS cell neuronal differentiation can be induced by coculturing the iPS cells with PA6 cells for 3-5 weeks under the following differentiation conditions: DMEM/F12 Glutamax (GIBCO), 10% knockout serum replacement (GIBCO), 0.1 mM nonessential amino acids (GIBCO), and 0.1 mM 2-mercaptoethanol (GIBCO). Alkaline phosphatase activity can be measured using the Vector Red Alkaline Phosphatase substrate kit I from Vector Laboratories. Neuronal differentiation can be monitored by immunostaining with various neuronal cell markers.

### GABA_{A} Receptor Agonists

As noted above, a subject method generally involves administering to an individual in need thereof an effective amount of a GABA_{A} receptor agonist. As used herein the term “GABA_{A} receptor agonist” includes compounds that bind to the GABA site (the site where GABA normally binds, also referred to as the “active” or “orthosteric” site) on a GABA_{A} receptor and activate the GABA_{A} receptor, resulting in increased CF conductance. As used herein the term “GABA_{A} receptor agonist” includes positive allosteric modulators of a GABA_{A} receptor, e.g., compounds that bind to allosteric sites on a GABA_{A} receptor complex and affect the GABA_{A} receptor in a positive manner, causing increased efficiency of the main site and therefore an indirect increase in Cl⁻ conductance. Non-limiting examples of GABA_{A} receptor agonists that bind to the GABA site include gaboxadol, ibotenic acid, muscimol, and progabide. Non-limiting examples of GABA_{A} receptor agonists that are positive allosteric modulators include barbiturates, benzodiazepines, carisoprodol, etodimate, glutethimide, kavalactones, meprobamate, neuroactive steroids, nonbenzodiazepines, propofol, theanine, and valerenic acid.

Non-limiting examples of GABA_{A} receptor agonists useful in methods described herein include triazolophthalazine derivatives, such as those disclosed in WO 99/25353, and WO/98/04560; tricyclic pyrazolo-pyridazinone analogs, such as those disclosed in WO 99/00391; fenamates, such as those disclosed in U.S. Pat. No. 5,637,617; triazolo-pyridazine derivatives, such as those disclosed in WO 99/37649, WO 99/37648, and WO 99/37644; pyrazolo-pyridine derivatives, such as those disclosed in WO 99/48892; nicotinic derivatives, such as those disclosed in WO 99/43661 and U.S. Pat. No. 5,723,462; muscimol, thiomuscimol, and compounds disclosed in U.S. Pat. No. 3,242,190; baclofen and compounds disclosed in U.S. Pat. No. 3,471,548; quisqualamine; ZAPA ((Z)-3-[(aminoiminomethyl)thio]-2-propenoic acid hydrochloride); zaleplon; THIP; imidazole-4-acetic acid (IMA); gabalinoleamide; isoguvicaine; 3-aminopropane sulphonic acid; piperidine-4-sulphonic acid (also known as P4S); 4,5,6,7-tetrahydro-[5,4-c]-pyridin-3-ol (also known as THIP or gaboxadol); 5-(4-piperidyl)-3-isothiazolol (also known as thio-4-PIOL); SR 95531; RU5315; CGP 55845; CGP 35348; FG 8094; SCH 50911; NG2-73; NGD-96-3; or picrotoxin and other bicyclophosphates disclosed in Bowery et al., Br. J. Pharmacol., 57; 435 (1976).

Also suitable for use is a heterocyclic compound that functions as a GABA_{A} receptor agonist and is described in U.S. Pat. No. 7,432,283. Also suitable for use is a GABA_{A} receptor agonist that is a substituted quinolone carboxylic acid, as described in U.S. Pat. No. 7,355,047. Also suitable for use are GABA_{A} receptor agonists that are aryl acid pyrimidinyl methyl amides, pyridazinyl methyl amides and related compounds of Formula I, as described in U.S. Pat. No. 7,351,826. Also suitable for use is a GABA_{A} receptor agonist that is a heteroaryl fused aminoalkyl imidazole derivative as described in U.S. Pat. No. 7,348,326. Also suitable for use is a GABA_{A} receptor agonist that is a bicyclic or tricyclic heteroaromatic compound as described in U.S. Pat. No. 7,348,326. Also suitable for use is a GABA_{A} receptor agonist that is a benzimidazole, a pyridylimidazole, or a related bicyclic heteroaryl compound of Formula I as described in U.S. Pat. No. 7,300,945. Also suitable for use is a GABA_{A} receptor agonist that is a substituted fused pyrroleoxime or a substituted fused pyrazoleoxime as described in U.S. Pat. No. 7,282,498. Also suitable for use is a GABA_{A} receptor agonist that is an imidazo-pyrimidine or a triazolo-pyrimidine as described in U.S. Pat. No. 7,271,170. Also suitable for use is a GABA_{A} receptor agonist that is an imidazol-1-ylmethyl pyridazine derivative as described in U.S. Pat. No. 7,122,546. Also suitable for use is a GABA_{A} receptor agonist that is a substituted imidazole derivative, as described in U.S. Pat. No. 7,030,144. Also suitable for use is a GABA_{A} receptor agonist that is an (Oxo-pyrazolo[1,5a]pyrimidin-2-yl)alkyl-carboxamide, as described in U.S. Pat. No. 7,008,947. Also suitable for use is a GABA_{A} receptor agonist that is a 4-imidazol-1-ylmethyl-pyrimidine derivative, as described in U.S. Pat. No. 6,951,864.

Additional non-limiting examples of suitable GABA_{A} receptor agonists include GABA_{A} receptor agonists described in U.S. Pat. Nos. 6,503,925; 6,218,547; 6,399,604; 6,646,124; 6,515,140; 6,451,809; 6,448,259; 6,448,246; 6,423,711; 6,414,147; 6,399,604; 6,380,209; 6,353,109; 6,297,256; 6,297,252; 6,268,496; 6,211,365; 6,166,203; 6,177,569; 6,194,427; 6,156,898; 6,143,760; 6,127,395; 6,103,903; 6,103,731; 6,723,735; 6,479,506; 6,476,030; 6,337,331; 6,730,676; 6,730,681; 6,828,322; 6,872,720; 6,699,859; 6,696,444; 6,617,326; 6,608,062; 6,579,875; 6,541,484; 6,500,828; 6,355,798; 6,333,336; 6,319,924; 6,303,605; 6,303,597; 6,291,460; 6,255,305; 6,133,255; 6,872,731; 6,900,215; 6,642,229; 6,593,325; 6,914,060; 6,914,063; 6,914,065; 6,936,608; 6,534,505; 6,426,343; 6,313,125; 6,310,203; 6,200,975; 6,071,909; 5,922,724; 6,096,887; 6,080,873; 6,013,799; 5,936,095; 5,925,770; 5,910,590; 5,908,932; 5,849,927; 5,840,888; 5,817,813; 5,804,686; 5,792,766; 5,750,702; 5,744,603; 5,744,602; 5,723,462; 5,696,260; 5,693,801; 5,677,309; 5,668,283; 5,637,725; 5,637,724; 5,625,063; 5,610,299; 5,608,079; 5,606,059; 5,604,235; 5,585,490; 5,510,480; 5,484,944; 5,473,073; 5,463,054; 5,451,585; 5,426,186; 5,367,077; 5,328,912; 5,326,868; 5,312,822; 5,306,819; 5,286,860; 5,266,698; 5,243,049; 5,216,159; 5,212,310; 5,185,446; 5,185,446; 5,182,290; 5,130,430; 5,095,015; or U.S. Patent Publication Nos. 20050014939; 20040171633; 20050165048; 20050165023; 20040259818; or 20040192692.

In some embodiments, the GABA_{A} receptor agonist is a subunit-selective modulator. Non-limiting examples of GABA_{A} receptor agonist having specificity for the alpha1 subunit include alpidem and zolpidem. Non-limiting examples of GABA_{A} receptor agonists having specificity for the alpha2 and/or alpha3 subunits include compounds described in U.S. Pat. Nos. 6,730,681; 6,828,322; 6,872,720; 6,699,859; 6,696,444; 6,617,326; 6,608,062; 6,579,875; 6,541,484; 6,500,828; 6,355,798; 6,333,336; 6,319,924; 6,303,605; 6,303,597; 6,291,460; 6,255,305; 6,133,255; 6,900,215; 6,642,229; 6,593,325; and 6,914,063. Non-limiting examples of GABA_{A} receptor agonist having specificity for the alpha2, alpha3 and/or alpha5 subunits include compounds described in U.S. Pat. Nos. 6,730,676 and 6,936,608. Non-limiting examples of GABA_{A} receptor agonists having specificity for the alpha5 subunit include compounds described in U.S. Pat. Nos. 6,534,505; 6,426,343; 6,313,125; 6,310,203; 6,200,975 and 6,399,604. Additional non-limiting subunit selective GABA_{A} receptor agonist include CL218,872 and related compounds disclosed in Squires et al., Pharmacol. Biochem. Behav., 10: 825 (1979); and beta-carboline-3-carboxylic acid esters described in Nielsen et al., Nature, 286: 606 (1980).

In some embodiments, a GABA_{A} receptor agonist is a positive allosteric modulator. In various embodiments, allosteric modulators modulate one or more aspects of the activity of GABA at the target GABA receptor, such as potency, maximal effect, affinity, and/or responsiveness to other GABA modulators. A positive allosteric modulator can potentiate the effect of GABA. Non-limiting examples of benzodiazepine GABA_{A} receptor agonists include aiprazolam, bentazepam, bretazenil, bromazepam, brotizolam, cannazepam, chlordiazepoxide, clobazam, clonazepam, cinolazepam, clotiazepam, cloxazolam, clozapin, delorazepam, diazepam, dibenzepin, dipotassium chlorazepat, divaplon, estazolam, ethyl-loflazepat, etizolam, fludiazepam, flunitrazepam, flurazepam 1HCl, flutoprazepam, halazeparn, haloxazolam, imidazenil, ketazolam, lorazepam, loprazolam, lormetazepam, medazepam, metaclazepam, mexozolam, midazolam-HCl, nabanezil, nimetazepam, nitrazepam, nordazepam, oxazepam-tazepam, oxazolam, pinazepam, prazepam, quazepam, suriclone, temazepam, tetrazepam, tofisopam, triazolam, zaleplon, zolezepam, zolpidem, zopiclone, and zopielon.

Additional non-limiting examples of benzodiazepine GABA_{A} receptor agonists include Rol5-4513, CL218872, CGS 8216, CGS 9895, PK 9084, U-93631, beta-CCM, beta-CCB, beta-CCP, Ro 19-8022, CGS 20625, NNC 14-0590, Ru 33-203, 5-amino-1-bromouracil, GYKI-52322, FG 8205, Ro 19-4603, ZG-63, RWJ46771, SX-3228, and L-655,078; NNC 14-0578, NNC 14-8198, and additional compounds described in Wong et al., Eur J Pharmacol 209: 319-325 (1995); Y-23684 and additional compounds in Yasumatsu et al., Br J Pharmacol 111: 1170-1178 (1994); and compounds described in U.S. Pat. No. 4,513,135.

Non-limiting examples of barbiturate or barbituric acid derivative GABA_{A} receptor agonists include phenobarbital, pentobarbital, pentobarbitone, primidone, barbexaclon, dipropyl barbituric acid, eunarcon, hexobarbital, mephobarbital, methohexital, Na-methohexital, 2,4,6(1H,3H,5)-pyrimidintrion, secbutabarbital and/or thiopental

Non-limiting examples of neurosteroid GABA_{A} receptor agonists include alphaxalone, allotetrahydrodeoxycorticosterone, tetrahydrodeoxycorticosterone, estrogen, progesterone 3-beta-hydroxyandrost-5-en-17-on-3-sulfate, dehydroepianrosterone, eltanolone, ethinylestradiol, 5-pregnen-3-beta-ol-20 on-sulfate, 5a-pregnan-3α-ol-20-one (5PG), allopregnanolone, pregnanolone, and steroid derivatives and metabolites described in U.S. Pat. Nos. 5,939,545, 5,925,630, 6,277,838, 6,143,736, RE35,517, U.S. Pat. Nos. 5,925,630, 5,591,733, 5,232,917, 20050176976, WO 96116076, WO 98/05337, WO 95/21617, WO 94/27608, WO 93/18053, WO 93/05786, WO 93/03732, WO 91116897, EP01038880, and Han et al., J. Med. Chem., 36, 3956-3967 (1993), Anderson et al., J. Med. Chem., 40, 1668-1681 (1997), Hogenkamp et al., J. Med. Chem., 40, 61-72 (1997), Upasani et al., J. Med. Chem., 40, 73-84 (1997), Majewska et al., Science 232:1004-1007 (1986), Harrison et al., J. Pharmacol. Exp. Ther. 241:346-353 (1987), Gee et al., Eur. J. Pharmacol., 136:419-423 (1987) and Birtran et al., Brain Res., 561, 157-161 (1991).

Non-limiting examples of beta-carboline GABA_{A} receptor agonists include abecarnil, 3,4-dihydro-beta-carboline, gedocarnil, 1-methyl-1-vinyl-2,3,4-trihydro-beta-carboline-3-carboxylic acid, 6-methoxy-1,2,3,4-tetrahydro-beta-carboline, N-BOC-L-1,2,3,4-tetrahydro-beta-carboline-3-carboxylic acid, tryptoline, pinoline, methoxyharmalan, tetrahydro-beta-carboline (THBC), 1-methyl-THBC, 6-methoxy-THBC, 6-hydroxy-THBC, 6-methoxyharmalan, norharman, 3,4-dihydro-beta-carboline, and compounds described in Nielsen et al., Nature, 286: 606 (1980).

In some embodiments, the GABA_{A} receptor agonist is the GABA-A agonist isoguvacine, which is described, e.g., in Chebib et al., Clin. Exp. Pharamacol. Physiol. 1999, 26, 937-940; Leinekugel et al. J. Physiol. 1995, 487, 319-29; and White et al., J. Neurochem. 1983, 40(6), 1701-8. In general, a total daily dose range for isoguvacine is from about 1 mg to about 2000 mg, or between about 5 mg to about 1000 mg.

In some embodiments, the GABA_{A} receptor agonist is the GABA_{A} receptor agonist gaboxadol (THIP; 4,5,6,7-tetrahydroisoxazolo[4,5-c]pyridine-3-ol), which is described, e.g., in U.S. Pat. No. 4,278,676 and Krogsgaard-Larsen, Acta. Chem. Scand. 1977, 31, 584. In general, a total daily dose range for gaboxadol is from about 1 mg to about 90 mg, or between about 2 mg to about 40 mg.

In some embodiments, the GABA_{A} receptor agonist is the GABA_{A} receptor agonist muscimol, which is described, e.g., in U.S. Pat. Nos. 3,242,190 and 3,397,209. In general, a total daily dose range for muscimol is from about 1 mg to about 2000 mg, or between about 5 mg to about 1000 mg.

In some embodiments, the GABA_{A} receptor agonist is the inverse GABA_{A} receptor agonist propyl-beta-carboline-3-carboxylate (beta-CCP), which is described, e.g., in Nielsen et al., J. Neurochem., 36(1):276-85 (1981). In general, a total daily dose range is from about 1 mg to about 2000 mg, or between about 5 mg to about 1000 mg.

“EC₅₀” values are concentrations of a GABA_{A} receptor agonist that promote the activity of a GABA receptor to half-maximal level. Methods for determining GABA_{A} receptor agonist activity, EC₅₀ values, binding affinities, target selectivity, physiological effects, mechanisms of action, and/or other aspects of GABA modulators are known in the art, and are described, e.g., in U.S. Pat. Nos. 6,737,242, 6,689,585, 6,586,582, 6,455,276, and 6,743,789.

A GABA_{A} receptor agonist used in methods described herein can have an EC₅₀ value with respect to one or more target GABA receptors of less than about 10 μM, or less than about or less than about 0.1 μM. In some embodiments, the GABA modulator has an EC₅₀ of less than about 50 nM, or less than about 10 nM, or less than about 1 nM. A suitable GABA_{A} receptor agonist can have an EC₅₀ of from about 1 nM to about 5 nM, from about 5 nM to about 10 nM, from about 10 nM to about 25 nM, from about 25 nM to about 50 nM, from about 50 nM to about 100 nM, from about 100 nM to about 500 nM, from about 500 nM to about 1 μM, from about 1 μM to about 5 μM, from about 5 μM to about 10 μM, from about 10 μM to about 25 μM, from about 25 μM to about 50 μM, or from about 50 μM to about 100 μM. In some embodiments, administration of a GABA_{A} receptor agonist according to methods described herein increases GABA activity within a target tissue by at least about 10%, at least about 15%, at least about 20%, at least about 25%, at least about 50%, at least about 75%, at least about 2-fold, at least about 2.5-fold, at least about 5-fold, or at least about 10-fold, or more than 10-fold. In some embodiments, the GABA_{A} receptor agonist has the desired activity at a concentration that is lower than the concentration of the GABA_{A} receptor agonist that is required to produce another, unrelated biological effect. In some cases, the concentration of the GABA_{A} receptor agonist required for GABA modulatory activity is at least 2-fold lower, or at least 5-fold lower, or at least 10-fold lower, or at least 20-fold lower than the concentration required to produce an unrelated biological effect.

In some embodiments, a GABA_{A} receptor agonist has “target selective” activity under certain conditions, wherein the GABA_{A} receptor agonist is substantially inactive against non-GABA molecular targets, such as (i) CNS receptors, including but not limited to, glutamate receptors, opioid receptors (e.g., mu, delta, and kappa opioid receptors), muscarinic receptors (e.g., m1-m5 receptors), histaminergic receptors, phencyclidine receptors, dopamine receptors, alpha and beta-adrenoceptors, sigma receptors (type-1 and type-2), and 5HT-1 and 5-HT-2 receptors; (ii) kinases, including but not limited to, Mitogen-activated protein kinase, PKA, PKB, PKC, CK-2; c-Met, JAK, SYK, KDR, FLT-3, c-Kit, Aurora kinase, CDK kinases (e.g., CDK4/cyclin D, CDK2/cyclin E, CDK2/cyclin A, CDK1/cyclin B), and TAK-1; (iii) non-GABA regulated ion channels (e.g., calcium, chloride, potassium, and the like) and/or (iv) enzymes, including but not limited to, histone deacetylases, phosphodiesterases, and the like. However, in other embodiments, GABA agent(s) are active against one or more additional receptors.

### Additional Agents

As noted above, a subject method can involve administration of one or more of: a GABA_{A} receptor agonist; an agent that stimulates or enhances release of GABA, e.g., from a GABAergic interneuron; an agent that inhibits GABA-transaminase; and a selective GABA reuptake inhibitor. Agents that stimulate or enhance release of GABA include, e.g., gabapentin (2-[1-(aminomethyl)cyclohexyl]acetic acid). Agents that inhibit GABA-transaminase include, e.g., γ-vinyl-GABA (GVG), also known as (RS)-4-aminohex-5-enoic acid. Agents that that are selective inhibitors of GABA reuptake (SGRI) include, e.g., tiagabine ((R)-1-[4,4-bis(3-methylthiophen-2-yl)but-3-enyl]piperidine-3-carboxylic acid).

### Crossing the Blood-Brain Barrier

Where a GABA_{A} receptor agonist (or other therapeutic agent) does not readily cross the blood-brain barrier (BBB), various methods can be used to facilitate crossing of the BBB, or circumventing the BBB altogether. Examples of methods of crossing the BBB include: use of vasoactive substances such as bradykinin or a bradykinin analog (where bradykinin analogs include, e.g., [Phe⁸ψ(CH₂—NH)Arg⁹]-bradykinin, N-acetyl-[Phe⁸ψ(CH₂—NH)Arg⁹]-bradykinin, desArg⁹-bradykinin, etc.); use of nitric oxide (NO) donor drugs (see below); localized exposure to high-intensity focused ultrasound; use of endogenous transport systems, including carrier-mediated transporters such as glucose and amino acid carriers; use of liposomes loaded with nanoparticles containing an agent (e.g., a GABA_{A} receptor agonist, where an example of such a nanoparticle is a polyethylene glycol-coated hexadecylcyanoacrylate nanosphere (see, e.g., Silva (2008) BMC Neurosci. 9:S4; Brigger et al. (2002) J. Pharm. Exp. Ther. 303:928; Wong et al. (2009) Adv. Drug Del. Rev. PMID 19914319; Khalil and Mainardes (2009) Curr. Drug. Del. 6:261; Modi et al. (2009) Prog. Neurobiol. 88:272; Barbu et al. (2009) Expert Opin. Drug. Del. 6:553); use of agents (e.g., Tariquidar) that inhibit P-glycoprotein at the BBB; and the like.

Suitable NO donor drugs include, e.g., organic nitrate compounds which are nitric acid esters of mono- and polyhydric alcohols, (e.g., glyceryl trinitrate (GTN) or nitroglycerin (NTG), pentaerythrityl tetranitrate (PETN), isosorbide dinitrate (ISDN), and isosorbide 5-mononitrate (IS-5-N)), S-nitrosothiol compounds (e.g., S-nitroso-N-acetyl-D,L-penicillamine (SNAP), S-nitrosoglutathione (SNOG), S-nitrosoalbumin, S-nitrosocysteine), sydnonimine compounds (e.g., molsidomine (N-ethoxycarbonyl-3-morpholino-sydnonimine), linsidomine (e.g., SIN-1; 3-morpholino-sydnonimine or 3-morpholinylsydnoneimine or 5-amino-3-morpholinyl-1,2,3-oxadiazolium), and pirsidomine (CAS 936).

In other embodiments, a GABA_{A} receptor agonist and/or other agent(s) of a combination is conjugated to a targeting domain to form a chimeric therapeutic, where the targeting domain facilitates passage of the blood-brain barrier (as described above) and/or binds one or more molecular targets in the CNS. In some embodiments, the targeting domain binds a target that is differentially expressed or displayed on, or in close proximity to, tissues, organs, and/or cells of interest. In some cases, the target is preferentially distributed in a neurogenic region of the brain, such as the dentate gyrus and/or the SVZ. For example, in some embodiments, a GABA_{A} receptor agonist and/or other agent(s) of a combination is conjugated or complexed with the fatty acid docosahexaenoic acid (DHA), which is readily transported across the blood brain barrier and imported into cells of the CNS.

### Further Combination Therapies

In some embodiments, a GABA_{A} receptor agonist is administered in combination therapy with at least one additional therapeutic agent. In some embodiments, a GABA_{A} receptor agonist is administered in conjunction with a stem cell therapy; and at least one additional therapeutic agent.

Suitable additional therapeutic agents include, but are not limited to, acetylcholinesterase inhibitors, including, but not limited to, Aricept (donepezil), Exelon (rivastigmine), metrifonate, and tacrine (Cognex); non-steroidal anti-inflammatory agents, including, but not limited to, ibuprofen and indomethacin; cyclooxygenase-2 (Cox2) inhibitors such as Celebrex; and monoamine oxidase inhibitors, such as Selegilene (Eldepryl or Deprenyl). Dosages for each of the above agents are known in the art. For example, Aricept is generally administered at 50 mg orally per day for 6 weeks, and, if well tolerated by the individual, at 10 mg per day thereafter.

Another suitable additional therapeutic agent is an apoE4 “structure corrector” that reduces apoE4 domain interaction. Agents that reduce apoE4 domain interaction include, e.g., an agent as described in U.S. Patent Publication No. 2006/0073104); and in Ye et al. (2005) Proc. Natl. Acad. Sci. USA 102:18700.

### Formulations, Dosages, and Routes of Delivery

An agent active agent (e.g., a GABA_{A} receptor agonist; at least a second therapeutic agent) can be provided together with a pharmaceutically acceptable excipient. Pharmaceutically acceptable excipients are known to those skilled in the art, and have been amply described in a variety of publications, including, for example, A. Gennaro (1995) “Remington: The Science and Practice of Pharmacy”, 19th edition, Lippincott, Williams, & Wilkins. In the discussion, below, of formulations, dosages, and routes of delivery, an “active agent” will refer to a GABA_{A} receptor agonist and/or at least a second therapeutic agent, unless otherwise specified.

An active agent (e.g., a GABA_{A} receptor agonist; at least a second therapeutic agent) can be incorporated into a variety of formulations for therapeutic administration. More particularly, an active agent can be formulated into pharmaceutical compositions by combination with appropriate pharmaceutically acceptable carriers or diluents, and may be formulated into preparations in solid, semi-solid, liquid or gaseous forms, such as, powders, granules, solutions, injections, inhalants, gels, hydrogels, microspheres, etc. As such, administration of an active agent can be achieved in various ways, including local, such as delivery into the affected tissue, oral, catheter mediated, intrathecal, buccal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation.

In some embodiments, an active agent(s) is formulated to cross the blood brain barrier (BBB). One strategy for drug delivery through the blood brain barrier (BBB) entails disruption of the BBB, either by osmotic means such as mannitol or leukotrienes, or biochemically by the use of vasoactive substances such as bradykinin. A BBB disrupting agent can be co-administered with an active agent when the compositions are administered by intravascular injection. Other strategies to go through the BBB may entail the use of endogenous transport systems, including carrier-mediated transporters such as glucose and amino acid carriers, receptor-mediated transcytosis for insulin or transferrin, and active efflux transporters such as p-glycoprotein. Active transport moieties may also be conjugated to an active agent for use in the methods disclosed herein to facilitate transport across the epithelial wall of the blood vessel. Alternatively, drug delivery behind the BBB is by intrathecal delivery of therapeutics directly to the cranium, as through an Ommaya reservoir.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, phosphate buffered saline (PBS), Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see. Langer, Science 249:1527-1533 (1990).

The pharmaceutical compositions can be administered for prophylactic and/or therapeutic treatments. Toxicity and therapeutic efficacy of the active agent can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred.

The data obtained from cell culture and/or animal studies can be used in formulating a range of dosages for humans. The dosage of the active agent typically lines within a range of circulating concentrations that include the ED50 with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under Good Manufacturing Practice (GMP) conditions.

The effective amount of an active agent(s) to be given to a particular patient will depend on a variety of factors, several of which will be different from patient to patient. A competent clinician will be able to determine an effective amount of an active agent to administer to a patient to treat an apoE4-associated disorder. Utilizing LD50 animal data, and other information available for the inhibitor, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

### Formulations

In carrying out a subject treatment method, an active agent(s) (e.g., a GABA_{A} receptor agonist; at least a second therapeutic agent) can be administered to the host using any convenient means capable of resulting in the desired physiological effect (e.g., increase in number of mature neurons; increase in functionality of neurons; increase in cognitive function; etc.). Thus, an active agent (e.g., a GABA_{A} receptor agonist; at least a second therapeutic agent) can be incorporated into a variety of formulations for therapeutic administration. More particularly, an active agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, an active agent(s) can be administered in the form of its (their) pharmaceutically acceptable salts, or the active agent may also be used alone or in appropriate association, as well as in combination, with other pharmaceutically active compounds. The following methods and excipients are merely exemplary and are in no way limiting.

For oral preparations, an active agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

An active agent can be formulated into preparations for injection by dissolving, suspending or emulsifying the active agent in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

An active agent can be utilized in aerosol formulation to be administered via inhalation. The compounds of the present invention can be formulated into pressurized acceptable propellants such as dichlorodifluoromethane, propane, nitrogen and the like.

Furthermore, an active agent can be made into suppositories by mixing with a variety of bases such as emulsifying bases or water-soluble bases. An active agent can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more active agents. Similarly, unit dosage forms for injection or intravenous administration may comprise the active agent(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of an active agent calculated in an amount sufficient to produce the desired effect in association with a pharmaceutically acceptable diluent, carrier or vehicle. The specifications for a unit dosage form of an active agent depend on the particular active agent employed and the effect to be achieved, and the pharmacodynamics associated with each active agent in the host.

Other modes of administration will also find use with the subject invention. For instance, an active agent can be formulated in suppositories and, in some cases, aerosol and intranasal compositions. For suppositories, the vehicle composition will include traditional binders and carriers such as, polyalkylene glycols, or triglycerides. Such suppositories may be formed from mixtures containing the active ingredient in the range of about 0.5% to about 10% (w/w), e.g., about 1% to about 2%.

Intranasal formulations will usually include vehicles that neither cause irritation to the nasal mucosa nor significantly disturb ciliary function. Diluents such as water, aqueous saline or other known substances can be employed. The nasal formulations may also contain preservatives such as, but not limited to, chlorobutanol and benzalkonium chloride. A surfactant may be present to enhance absorption of an active agent by the nasal mucosa.

An active agent can be administered in an injectable form, e.g., the active agent can be in a formulation suitable for injection (e.g., intravenous injection, intramuscular injection, subcutaneous injection, intrathecal injection, etc.). Injectable compositions can be prepared as liquid solutions or suspensions; solid forms suitable for solution in, or suspension in, liquid vehicles prior to injection may also be prepared. The preparation may also be emulsified or the active agent encapsulated in liposome vehicles.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the active agent adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

### Oral Formulations

In some embodiments, an active agent is formulated for oral delivery to an individual in need of such an agent.

For oral delivery, a formulation comprising an active agent will in some embodiments include an enteric-soluble coating material. Suitable enteric-soluble coating material include hydroxypropyl methylcellulose acetate succinate (HPMCAS), hydroxypropyl methyl cellulose phthalate (HPMCP), cellulose acetate phthalate (CAP), polyvinyl phthalic acetate (PVPA), Eudragit™, and shellac.

As one non-limiting example of a suitable oral formulation, an active agent is formulated with one or more pharmaceutical excipients and coated with an enteric coating, as described in U.S. Pat. No. 6,346,269. For example, a solution comprising an active agent and a stabilizer is coated onto a core comprising pharmaceutically acceptable excipients, to form an active agent-coated core; a sub-coating layer is applied to the active agent-coated core, which is then coated with an enteric coating layer. The core generally includes pharmaceutically inactive components such as lactose, a starch, mannitol, sodium carboxymethyl cellulose, sodium starch glycolate, sodium chloride, potassium chloride, pigments, salts of alginic acid, talc, titanium dioxide, stearic acid, stearate, micro-crystalline cellulose, glycerin, polyethylene glycol, triethyl citrate, tributyl citrate, propanyl triacetate, dibasic calcium phosphate, tribasic sodium phosphate, calcium sulfate, cyclodextrin, and castor oil. Suitable solvents for an active agent include aqueous solvents. Suitable stabilizers include alkali-metals and alkaline earth metals, bases of phosphates and organic acid salts and organic amines. The sub-coating layer comprises one or more of an adhesive, a plasticizer, and an anti-tackiness agent. Suitable anti-tackiness agents include talc, stearic acid, stearate, sodium stearyl fumarate, glyceryl behenate, kaolin and aerosil. Suitable adhesives include polyvinyl pyrrolidone (PVP), gelatin, hydroxyethyl cellulose (HEC), hydroxypropyl cellulose (HPC), hydroxypropyl methyl cellulose (HPMC), vinyl acetate (VA), polyvinyl alcohol (PVA), methyl cellulose (MC), ethyl cellulose (EC), hydroxypropyl methyl cellulose phthalate (HPMCP), cellulose acetate phthalates (CAP), xanthan gum, alginic acid, salts of alginic acid, Eudragit™, copolymer of methyl acrylic acid/methyl methacrylate with polyvinyl acetate phthalate (PVAP). Suitable plasticizers include glycerin, polyethylene glycol, triethyl citrate, tributyl citrate, propanyl triacetate and castor oil. Suitable enteric-soluble coating material include hydroxypropyl methylcellulose acetate succinate (HPMCAS), hydroxypropyl methyl cellulose phthalate (HPMCP), cellulose acetate phthalate (CAP), polyvinyl phthalic acetate (PVPA), Eudragit™ and shellac.

Suitable oral formulations also include an active agent, formulated with any of the following: microgranules (see, e.g., U.S. Pat. No. 6,458,398); biodegradable macromers (see, e.g., U.S. Pat. No. 6,703,037); biodegradable hydrogels (see, e.g., Graham and McNeill (1989) Biomaterials 5:27-36); biodegradable particulate vectors (see, e.g., U.S. Pat. No. 5,736,371); bioabsorbable lactone polymers (see, e.g., U.S. Pat. No. 5,631,015); slow release protein polymers (see, e.g., U.S. Pat. No. 6,699,504; Pelias Technologies, Inc.); a poly(lactide-co-glycolide/polyethylene glycol block copolymer (see, e.g., U.S. Pat. No. 6,630,155; Atrix Laboratories, Inc.); a composition comprising a biocompatible polymer and particles of metal cation-stabilized agent dispersed within the polymer (see, e.g., U.S. Pat. No. 6,379,701; Alkermes Controlled Therapeutics, Inc.); and microspheres (see, e.g., U.S. Pat. No. 6,303,148; Octoplus, B.V.).

Suitable oral formulations also include an active agent formulated with any of the following: a carrier such as Emisphere® (Emisphere Technologies, Inc.); TIMERx, a hydrophilic matrix combining xanthan and locust bean gums which, in the presence of dextrose, form a strong binder gel in water (Penwest); Geminex™ (Penwest); Procise™ (GlaxoSmithKline); SAVIT™ (Mistral Pharma Inc.); RingCap™ (Alza Corp.); Smartrix® (Smartrix Technologies, Inc.); SQZgel™ (MacroMed, Inc.); Geomatrix™ (Skye Pharma, Inc.); Oros® Tri-layer (Alza Corporation); and the like.

Also suitable for use are formulations such as those described in U.S. Pat. No. 6,296,842 (Alkermes Controlled Therapeutics, Inc.); U.S. Pat. No. 6,187,330 (Scios, Inc.); and the like.

Also suitable for use herein are formulations comprising an intestinal absorption enhancing agent. Suitable intestinal absorption enhancers include, but are not limited to, calcium chelators (e.g., citrate, ethylenediamine tetracetic acid); surfactants (e.g., sodium dodecyl sulfate, bile salts, palmitoylcarnitine, and sodium salts of fatty acids); toxins (e.g., zonula occludens toxin); and the like.

### Controlled Release Formulations

In some embodiments, an active agent is formulated in a controlled release formulation.

Controlled release formulations suitable for use can be taken to mean any one of a number of extended release dosage forms. The following terms may be considered to be substantially equivalent to controlled release, for the purposes of the present disclosure: continuous release, controlled release, delayed release, depot, gradual release, long-term release, programmed release, prolonged release, proportionate release, protracted release, repository, slow release, spaced release, sustained release, time coat, timed release, delayed action, extended action, layered-time action, long acting, prolonged action, repeated action, slowing acting, sustained action, sustained-action medications, and extended release. Further discussions of these terms may be found in Lesczek Krowczynski, Extended-Release Dosage Forms, 1987 (CRC Press, Inc.).

The various controlled release technologies cover a very broad spectrum of drug dosage forms. Controlled release technologies include, but are not limited to physical systems and chemical systems.

Physical systems include, but are not limited to, reservoir systems with rate-controlling membranes, such as microencapsulation, macroencapsulation, and membrane systems; reservoir systems without rate-controlling membranes, such as hollow fibers, ultra microporous cellulose triacetate, and porous polymeric substrates and foams; monolithic systems, including those systems physically dissolved in non-porous, polymeric, or elastomeric matrices (e.g., nonerodible, erodible, environmental agent ingression, and degradable), and materials physically dispersed in non-porous, polymeric, or elastomeric matrices (e.g., nonerodible, erodible, environmental agent ingression, and degradable); laminated structures, including reservoir layers chemically similar or dissimilar to outer control layers; and other physical methods, such as osmotic pumps, or adsorption onto ion-exchange resins.

Chemical systems include, but are not limited to, chemical erosion of polymer matrices (e.g., heterogeneous, or homogeneous erosion), or biological erosion of a polymer matrix (e.g., heterogeneous, or homogeneous). Additional discussion of categories of systems for controlled release may be found in Agis F. Kydonieus, Controlled Release Technologies: Methods, Theory and Applications, 1980 (CRC Press, Inc.).

There are a number of controlled release drug formulations that are developed for oral administration. These include, but are not limited to, osmotic pressure-controlled gastrointestinal delivery systems; hydrodynamic pressure-controlled gastrointestinal delivery systems; membrane permeation-controlled gastrointestinal delivery systems, which include microporous membrane permeation-controlled gastrointestinal delivery devices; gastric fluid-resistant intestine targeted controlled-release gastrointestinal delivery devices; gel diffusion-controlled gastrointestinal delivery systems; and ion-exchange-controlled gastrointestinal delivery systems, which include cationic and anionic drugs. Additional information regarding controlled release drug delivery systems may be found in Yie W. Chien, Novel Drug Delivery Systems, 1992 (Marcel Dekker, Inc.). Some of these formulations will now be discussed in more detail.

Enteric coatings are applied to tablets to prevent the release of drugs in the stomach either to reduce the risk of unpleasant side effects or to maintain the stability of the drug which might otherwise be subject to degradation of expose to the gastric environment. Polymers that are used for this purpose include polyacids that function by virtue or the fact that their solubility in aqueous medium is pH-dependent, and they require conditions with a pH higher than normally encountered in the stomach.

One exemplary type of oral controlled release structure is enteric coating of a solid or liquid dosage form. The enteric coatings are designed to disintegrate in intestinal fluid for ready absorption. Delay of absorption of the active agent that is incorporated into a formulation with an enteric coating is dependent on the rate of transfer through the gastrointestinal tract, and so the rate of gastric emptying is an important factor. Some investigators have reported that a multiple-unit type dosage form, such as granules, may be superior to a single-unit type. Therefore, in one exemplary embodiment, an active agent may be contained in an enterically coated multiple-unit dosage form. In an exemplary embodiment, an active agent dosage form is prepared by spray-coating granules of an active agent-enteric coating agent solid dispersion on an inert core material. These granules can result in prolonged absorption of the drug with good bioavailability.

Suitable enteric coating agents include, but are not limited to, hydroxypropylmethylcellulose phthalate, methacryclic acid-methacrylic acid ester copolymer, polyvinyl acetate-phthalate and cellulose acetate phthalate Akihiko Hasegawa, Application of solid dispersions of Nifedipine with enteric coating agent to prepare a sustained-release dosage form, Chem. Pharm. Bull. 33: 1615-1619 (1985). Various enteric coating materials may be selected on the basis of testing to achieve an enteric coated dosage form designed ab initio to have an optimal combination of dissolution time, coating thicknesses and diametral crushing strength. S. C. Porter et al., The Properties of Enteric Tablet Coatings Made From Polyvinyl Acetate-phthalate and Cellulose acetate Phthalate, J. Pharm. Pharmacol. 22:42 p (1970).

Another type of useful oral controlled release structure is a solid dispersion. A solid dispersion may be defined as a dispersion of one or more active ingredients in an inert carrier or matrix in the solid state prepared by the melting (fusion), solvent, or melting-solvent method Akihiko Hasegawa, Super Saturation Mechanism of Drugs from Solid Dispersions with Enteric Coating Agents, Chem. Pharm. Bull. 36: 4941-4950 (1998). The solid dispersions may be also called solid-state dispersions. The term “coprecipitates” may also be used to refer to those preparations obtained by the solvent methods.

The selection of the carrier may have an influence on the dissolution characteristics of the dispersed active agent because the dissolution rate of a component from a surface may be affected by other components in a multiple component mixture. For example, a water-soluble carrier may result in a fast release of the active agent from the matrix, or a poorly soluble or insoluble carrier may lead to a slower release of the active agent from the matrix. The solubility of the active agent may also be increased owing to some interaction with the carriers.

Examples of carriers useful in solid dispersions include, but are not limited to, water-soluble polymers such as polyethylene glycol, polyvinylpyrrolidone, and hydroxypropylmethylcellulose. Alternative carriers include phosphatidylcholine. Phosphatidylcholine is an amphoteric but water-insoluble lipid, which may improve the solubility of otherwise insoluble active agents in an amorphous state in phosphatidylcholine solid dispersions.

Other carriers include polyoxyethylene hydrogenated castor oil. Poorly water-soluble active agents may be included in a solid dispersion system with an enteric polymer such as hydroxypropylmethylcellulose phthalate and carboxymethylethylcellulose, and a non-enteric polymer, hydroxypropylmethylcellulose. Another solid dispersion dosage form includes incorporation of the active agent with ethyl cellulose and stearic acid in different ratios.

There are various methods commonly known for preparing solid dispersions. These include, but are not limited to, the melting method, the solvent method and the melting-solvent method.

Another controlled release dosage form is a complex between an ion exchange resin and an active agent. Ion exchange resin-drug complexes have been used to formulate sustained-release products of acidic and basic drugs. In one exemplary embodiment, a polymeric film coating is provided to the ion exchange resin-drug complex particles, making drug release from these particles diffusion controlled. See Y. Raghunathan et al., Sustained-released drug delivery system I: Coded ion-exchange resin systems for phenylpropanolamine and other drugs, J. Pharm. Sciences 70: 379-384 (1981).

Injectable microspheres are another controlled release dosage form. Injectable micro spheres may be prepared by non-aqueous phase separation techniques, and spray-drying techniques. Microspheres may be prepared using polylactic acid or copoly(lactic/glycolic acid). Shigeyuki Takada, Utilization of an Amorphous Form of a Water-Soluble GPIIb/IIIa Antagonist for Controlled Release From Biodegradable Micro spheres, Pharm. Res. 14:1146-1150 (1997), and ethyl cellulose, Yoshiyuki Koida, Studies on Dissolution Mechanism of Drugs from Ethyl Cellulose Microcapsules, Chem. Pharm. Bull. 35:1538-1545 (1987).

Other controlled release technologies that may be used include, but are not limited to, SODAS (Spheroidal Oral Drug Absorption System), INDAS (Insoluble Drug Absorption System), IPDAS (Intestinal Protective Drug Absorption System), MODAS (Multiporous Oral Drug Absorption System), EFVAS (Effervescent Drug Absorption System), PRODAS (Programmable Oral Drug Absorption System), and DUREDAS (Dual Release Drug Absorption System) available from Elan Pharmaceutical Technologies. SODAS are multi particulate dosage forms utilizing controlled release beads. INDAS are a family of drug delivery technologies designed to increase the solubility of poorly soluble drugs. IPDAS are multi particulate tablet formation utilizing a combination of high density controlled release beads and an immediate release granulate. MODAS are controlled release single unit dosage forms. Each tablet consists of an inner core surrounded by a semipermeable multiparous membrane that controls the rate of drug release. EFVAS is an effervescent drug absorption system. PRODAS is a family of multi particulate formulations utilizing combinations of immediate release and controlled release mini-tablets. DUREDAS is a bilayer tablet formulation providing dual release rates within the one dosage form. Although these dosage forms are known to one of skill, certain of these dosage forms will now be discussed in more detail.

INDAS was developed specifically to improve the solubility and absorption characteristics of poorly water soluble drugs. Solubility and, in particular, dissolution within the fluids of the gastrointestinal tract is a key factor in determining the overall oral bioavailability of poorly water soluble drug. By enhancing solubility, one can increase the overall bioavailability of a drug with resulting reductions in dosage.

IPDAS is a multi-particulate tablet technology that may enhance the gastrointestinal tolerability of potential irritant and ulcerogenic drugs. Intestinal protection is facilitated by the multi-particulate nature of the IPDAS formulation which promotes dispersion of an irritant lipoate throughout the gastrointestinal tract. Controlled release characteristics of the individual beads may avoid high concentration of active agent being both released locally and absorbed systemically. The combination of both approaches serves to minimize the potential harm of the active agent with resultant benefits to patients.

IPDAS is composed of numerous high density controlled release beads. Each bead may be manufactured by a two step process that involves the initial production of a micromatrix with embedded active agent and the subsequent coating of this micromatrix with polymer solutions that form a rate-limiting semipermeable membrane in vivo. Once an IPDAS tablet is ingested, it may disintegrate and liberate the beads in the stomach. These beads may subsequently pass into the duodenum and along the gastrointestinal tract, e.g., in a controlled and gradual manner, independent of the feeding state. Release of the active agent occurs by diffusion process through the micromatrix and subsequently through the pores in the rate controlling semipermeable membrane. The release rate from the IPDAS tablet may be customized to deliver a drug-specific absorption profile associated with optimized clinical benefit. Should a fast onset of activity be necessary, immediate-release granulate may be included in the tablet. The tablet may be broken prior to administration, without substantially compromising drug release, if a reduced dose is required for individual titration.

MODAS is a drug delivery system that may be used to control the absorption of water soluble agents. Physically MODAS is a non-disintegrating table formulation that manipulates drug release by a process of rate limiting diffusion by a semipermeable membrane formed in vivo. The diffusion process essentially dictates the rate of presentation of drug to the gastrointestinal fluids, such that the uptake into the body is controlled. Because of the minimal use of excipients, MODAS can readily accommodate small dosage size forms. Each MODAS tablet begins as a core containing active drug plus excipients. This core is coated with a solution of insoluble polymers and soluble excipients. Once the tablet is ingested, the fluid of the gastrointestinal tract may dissolve the soluble excipients in the outer coating leaving substantially the insoluble polymer. What results is a network of tiny, narrow channels connecting fluid from the gastrointestinal tract to the inner drug core of water soluble drug. This fluid passes through these channels, into the core, dissolving the drug, and the resultant solution of drug may diffuse out in a controlled manner. This may permit both controlled dissolution and absorption. An advantage of this system is that the drug releasing pores of the tablet are distributed over substantially the entire surface of the tablet. This facilitates uniform drug absorption reduces aggressive unidirectional drug delivery. MODAS represents a very flexible dosage form in that both the inner core and the outer semipermeable membrane may be altered to suit the individual delivery requirements of a drug. In particular, the addition of excipients to the inner core may help to produce a microenvironment within the tablet that facilitates more predictable release and absorption rates. The addition of an immediate release outer coating may allow for development of combination products.

Additionally, PRODAS may be used to deliver an active agent. PRODAS is a multi particulate drug delivery technology based on the production of controlled release mini tablets in the size range of 1.5 to 4 mm in diameter. The PRODAS technology is a hybrid of multi particulate and hydrophilic matrix tablet approaches, and may incorporate, in one dosage form, the benefits of both these drug delivery systems.

In its most basic form, PRODAS involves the direct compression of an immediate release granulate to produce individual mini tablets that contain an active agent. These mini tablets are subsequently incorporated into hard gels and capsules that represent the final dosage form. A more beneficial use of this technology is in the production of controlled release formulations. In this case, the incorporation of various polymer combinations within the granulate may delay the release rate of drugs from each of the individual mini tablets. These mini tablets may subsequently be coated with controlled release polymer solutions to provide additional delayed release properties. The additional coating may be necessary in the case of highly water soluble drugs or drugs that are perhaps gastroirritants where release can be delayed until the formulation reaches more distal regions of the gastrointestinal tract. One value of PRODAS technology lies in the inherent flexibility to formulation whereby combinations of mini tablets, each with different release rates, are incorporated into one dosage form. As well as potentially permitting controlled absorption over a specific period, this also may permit targeted delivery of drug to specific sites of absorption throughout the gastrointestinal tract. Combination products also may be possible using mini tablets formulated with different active ingredients.

DUREDAS is a bilayer tableting technology that may be used to formulate an active agent. DUREDAS was developed to provide for two different release rates, or dual release of a drug from one dosage form. The term bilayer refers to two separate direct compression events that take place during the tableting process. In an exemplary embodiment, an immediate release granulate is first compressed, being followed by the addition of a controlled release element which is then compressed onto this initial tablet. This may give rise to the characteristic bilayer seen in the final dosage form.

The controlled release properties may be provided by a combination of hydrophilic polymers. In certain cases, a rapid release of an active agent may be desirable in order to facilitate a fast onset of therapeutic affect. Hence one layer of the tablet may be formulated as an immediate-release granulate. By contrast, the second layer of the tablet may release the drug in a controlled manner, e.g., through the use of hydrophilic polymers. This controlled release may result from a combination of diffusion and erosion through the hydrophilic polymer matrix.

A further extension of DUREDAS technology is the production of controlled release combination dosage forms. In this instance, two different active agents may be incorporated into the bilayer tablet and the release of drug from each layer controlled to maximize therapeutic affect of the combination.

An active agent can be incorporated into any one of the aforementioned controlled released dosage forms, or other conventional dosage forms. The amount of active agent contained in each dose can be adjusted, to meet the needs of the individual patient, and the indication. One of skill in the art and reading this disclosure will readily recognize how to adjust the level of active agent and the release rates in a controlled release formulation, in order to optimize delivery of an active agent and its bioavailability.

### Inhalational Formulations

An active agent will in some embodiments be administered to a patient by means of a pharmaceutical delivery system for the inhalation route. An active agent may be formulated in a form suitable for administration by inhalation. The inhalational route of administration provides the advantage that the inhaled drug can bypass the blood-brain barrier. The pharmaceutical delivery system is one that is suitable for respiratory therapy by delivery of an active agent to mucosal linings of the bronchi. An active agent can be delivered by a system that depends on the power of a compressed gas to expel the active agent from a container. An aerosol or pressurized package can be employed for this purpose.

As used herein, the term “aerosol” is used in its conventional sense as referring to very fine liquid or solid particles carries by a propellant gas under pressure to a site of therapeutic application. When a pharmaceutical aerosol is employed in this invention, the aerosol contains an active agent, which can be dissolved, suspended, or emulsified in a mixture of a fluid carrier and a propellant. The aerosol can be in the form of a solution, suspension, emulsion, powder, or semi-solid preparation. Aerosols employed in the present invention are intended for administration as fine, solid particles or as liquid mists via the respiratory tract of a patient. Various types of propellants known to one of skill in the art can be utilized. Suitable propellants include, but are not limited to, hydrocarbons or other suitable gas. In the case of the pressurized aerosol, the dosage unit may be determined by providing a value to deliver a metered amount.

An active agent can also be formulated for delivery with a nebulizer, which is an instrument that generates very fine liquid particles of substantially uniform size in a gas. For example, a liquid containing an active agent is dispersed as droplets. The small droplets can be carried by a current of air through an outlet tube of the nebulizer. The resulting mist penetrates into the respiratory tract of the patient.

A powder composition containing an active agent, with or without a lubricant, carrier, or propellant, can be administered to a mammal in need of therapy. This embodiment can be carried out with a conventional device for administering a powder pharmaceutical composition by inhalation. For example, a powder mixture of the active agent and a suitable powder base such as lactose or starch may be presented in unit dosage form in for example capsular or cartridges, e.g. gelatin, or blister packs, from which the powder may be administered with the aid of an inhaler.

There are several different types of inhalation methodologies which can be employed in connection with the present disclosure. An active agent can be formulated in basically three different types of formulations for inhalation. First, an active agent can be formulated with low boiling point propellants. Such formulations are generally administered by conventional meter dose inhalers (MDI's). However, conventional MDI's can be modified so as to increase the ability to obtain repeatable dosing by utilizing technology which measures the inspiratory volume and flow rate of the patient as discussed within U.S. Pat. Nos. 5,404,871 and 5,542,410.

Alternatively, an active agent can be formulated in aqueous or ethanolic solutions and delivered by conventional nebulizers. In some embodiments, such solution formulations are aerosolized using devices and systems such as disclosed within U.S. Pat. Nos. 5,497,763; 5,544,646; 5,718,222; and 5,660,166.

An active agent can be formulated into dry powder formulations. Such formulations can be administered by simply inhaling the dry powder formulation after creating an aerosol mist of the powder. Technology for carrying such out is described within U.S. Pat. No. 5,775,320 issued Jul. 7, 1998 and U.S. Pat. No. 5,740,794 issued Apr. 21, 1998.

### Dosages

Although the dosage used will vary depending on the clinical goals to be achieved, a suitable dosage range is one which provides up to about 1 μg to about 1,000 μg or about 10,000 μg of an active agent and can be administered in a single dose. Alternatively, a target dosage of an active agent can be considered to be about in the range of about 0.1-1000 μM, about 0.5-500 μM, about 1-100 μM, or about 5-50 μM in a sample of host blood drawn within the first 24-48 hours after administration of the agent.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

In some embodiments, multiple doses of an active agent are administered. The frequency of administration of an active agent can vary depending on any of a variety of factors, e.g., severity of the symptoms, etc. For example, in some embodiments, an active agent is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid). In some embodiments, an active agent is administered continuously.

The duration of administration of an active agent, e.g., the period of time over which an active agent is administered, can vary, depending on any of a variety of factors, e.g., patient response, etc. For example, an active agent can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In some embodiments, an agent is administered for the remaining lifetime of the individual.

In some embodiments, administration of an active agent is discontinuous, e.g., an active agent is administered for a first period of time and at a first dosing frequency; administration of the active agent is suspended for a period of time; then the active agent is administered for a second period of time for a second dosing frequency. The period of time during which administration of the active agent is suspended can vary depending on various factors, e.g., cognitive functions of the individual; and will generally range from about 1 week to about 6 months, e.g., from about 1 week to about 2 weeks, from about 2 weeks to about 4 weeks, from about one month to about 2 months, from about 2 months to about 4 months, or from about 4 months to about 6 months, or longer. The first period of time may be the same or different than the second period of time; and the first dosing frequency may be the same or different than the second dosing frequency.

### Routes of Administration

An active agent is administered to an individual using any available method and route suitable for drug delivery, including in vivo and ex vivo methods, as well as systemic and localized routes of administration.

Conventional and pharmaceutically acceptable routes of administration include intranasal, intramuscular, intratracheal, subcutaneous, intradermal, topical application, intravenous, rectal, nasal, oral and other enteral and parenteral routes of administration. Routes of administration may be combined, if desired, or adjusted depending upon the agent and/or the desired effect. The active agent can be administered in a single dose or in multiple doses. In some embodiments, the active agent is administered orally. In other specific embodiments, the active agent is administered via an inhalational route. In some embodiments, the active agent is administered intranasally.

The active agent can be administered to a host using any available conventional methods and routes suitable for delivery of conventional drugs, including systemic or localized routes. In general, routes of administration contemplated by the present disclosure include, but are not necessarily limited to, enteral, parenteral, and inhalational routes.

Parenteral routes of administration other than inhalation administration include, but are not necessarily limited to, topical, transdermal, subcutaneous, intramuscular, intraorbital, intracapsular, intraspinal, intrasternal, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

The active agent can also be delivered to the subject by enteral administration. Enteral routes of administration include, but are not necessarily limited to, oral and rectal (e.g., using a suppository) delivery.

Methods of administration of the active agent through the skin or mucosa include, but are not necessarily limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” which deliver their product continuously via electric pulses through unbroken skin for periods of several days or more.

### Stem Cells

For administration to a mammalian host, an NSC population can be formulated as a pharmaceutical composition. A pharmaceutical composition can be a sterile aqueous or non-aqueous solution, suspension or emulsion, which additionally comprises a physiologically acceptable carrier (i.e., a non-toxic material that does not interfere with the activity of the active ingredient). Any suitable carrier known to those of ordinary skill in the art may be employed in a subject pharmaceutical composition. Representative carriers include physiological saline solutions, gelatin, water, alcohols, natural or synthetic oils, saccharide solutions, glycols, injectable organic esters such as ethyl oleate or a combination of such materials. Optionally, a pharmaceutical composition may additionally contain preservatives and/or other additives such as, for example, antimicrobial agents, anti-oxidants, chelating agents and/or inert gases, and/or other active ingredients.

For example, an NSC population can be supplied in the form of a pharmaceutical composition comprising an isotonic excipient prepared under sufficiently sterile conditions for human administration. For general principles in medicinal formulation, see, e.g., Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000.

In some embodiments, an NSC population is encapsulated, according to known encapsulation technologies, including microencapsulation (see, e.g., U.S. Pat. Nos. 4,352,883; 4,353,888; and 5,084,350). Where the NSCs are encapsulated, in some embodiments the NSCs are encapsulated by macroencapsulation, as described in U.S. Pat. Nos. 5,284,761; 5,158,881; 4,976,859; 4,968,733; 5,800,828 and published PCT patent application WO 95/05452.

A unit dosage form of an NSC population can contain from about 10³ cells to about 10⁹ cells, e.g., from about 10³ cells to about 10⁴ cells, from about 10⁴ cells to about 10⁵ cells, from about 10⁵ cells to about 10⁶ cells, from about 10⁶ cells to about 10⁷ cells, from about 10⁷ cells to about 10⁸ cells, or from about 10⁸ cells to about 10⁹ cells.

An NSC population will in some embodiments be transplanted into a patient according to conventional techniques, into the CNS, as described for example, in U.S. Pat. Nos. 5,082,670 and 5,618,531, or into any other suitable site in the body. In one embodiment, a population of NSCs is transplanted directly into the CNS. Parenchymal and intrathecal sites are also suitable. It will be appreciated that the exact location in the CNS will vary according to the disease state. Cells may be introduced by, for example, stereotaxic implantation or intracerebral grafting into the CNS of a patient.

In some embodiments, an NSC population is administered as a cell suspension. In other embodiments, an NSC population is administered as neurospheres. In other embodiments, an NSC population is administered in an encapsulated form. In other embodiments, an NSC population is contained with a reservoir, and the reservoir is implanted into the individual.

A single dose of an NSC population can contain from about 10³ cells to about 10⁹ cells, e.g., from about 10³ cells to about 10⁴ cells, from about 10⁴ cells to about 10⁵ cells, from about 10⁵ cells to about 10⁶ cells, from about 10⁶ cells to about 10⁷ cells, from about 10⁷ cells to about 10⁸ cells, or from about 10⁸ cells to about 10⁹ cells. In some embodiments, multiple doses of an NSC population are administered to an individual in need of such treatment. Doses can be administered at regular intervals (e.g., once a week, once a month, once every 6 weeks, once every 8 weeks, once every 6 months, etc.). Alternatively doses beyond an initial dose can be administered according to need, as determined by a medical professional, e.g., based on reappearance of symptoms associated with an apoE4-associated neurodegenerative disorder.

### Subjects Suitable for Treatment

A variety of subjects are suitable for treatment with a subject method. Suitable subjects include any individual, particularly a human, who has an apoE4-associated disorder, who is at risk for developing an apoE-associated disorder, who has had an apoE-associated disorder and is at risk for recurrence of the apoE4-associated disorder, or who is recovering from an apoE4-associated disorder.

Subjects suitable for treatment with a subject method include individuals who have one apoE4 allele; and individuals who have two apoE4 alleles. In other words, suitable subjects include those who are homozygous for apoE4 and those who are heterozygous for apoE4. For example, an individual can have an apoE3/apoE4 genotype, or an apoE4/apoE4 genotype. In some embodiments, the subject has been diagnosed as having Alzheimer's disease.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

Adult neurogenesis was investigated in mice with knockout (KO) for apoE or with knock-in (KI) alleles for human apoE3 or apoE4, and report that neurogenesis is reduced in both apoE-KO and apoE4-KI mice. In apoE-KO mice, increased BMP signaling promoted glial differentiation at the expense of neurogenesis. In contrast, in apoE4-KI mice presynaptic GABAergic input-mediated maturation of newborn neurons was diminished. Tau phosphorylation, an Alzheimer's disease characteristic, and levels of neurotoxic apoE fragments were both elevated in apoE4-KI hippocampal neurons concomitant with decreased GABAergic interneuron survival. Potentiating GABAergic signaling restored neuronal maturation and neurogenesis in apoE4-KI mice to normal levels. These findings indicate that GABAergic signaling can be targeted to mitigate the deleterious effects of apoE4 on neurogenesis.

### Materials and Methods

### Animals

Wildtype and apoE-KO mice were from the Jackson Laboratory (Bar Harbor, Me.). Human apoE3-KI and apoE4-KI mice, which were reported previously (Sullivan et al., 1997; Sullivan et al., 2004), were from Taconic (Hudson, N.Y.). GFAP-apoE3 and GFAP-apoE4 transgenic mice were generated at Gladstone (Brecht et al., 2004). The EGFP_{apoE} reporter mice were reported previously (Xu et al., 2006). The gene-targeting vector was constructed from a subclone of an 8.3-kb EcoR1 fragment spanning exons 1-4 of mouse Apoe isolated from a 129/SvJae mouse genomic bacterial artificial chromosome library (Invitrogen, Carlsbad, Calif.). Raffaï et al. (2001) Proc Natl Acad Sci USA 98, 11587-11591. EGFP cDNA with a stop codon was inserted into the mouse apoE gene locus, immediately after the translation initiation site in exon 2. All mice were on a pure C57/B6 genetic background. The mice were weaned at 21 days of age, housed in a barrier facility at the Gladstone Animal Core with a 12-h light/12-h dark cycle, and fed a chow diet containing 4.5% fat (Ralston Purina). All animal experiments were performed in compliance with standard governmental and NIH regulations.

### BrdU Injection and Collection of Mouse Brains

Female mice (3, 6-7, or 12-13 months of age) received two intraperitoneal injections of BrdU (Sigma, 100 mg/kg body weight) 6 h apart. One day, 3 days, and 4 and 10 weeks after the second injection, the brains were perfused with phosphate-buffered saline and collected.

Treatment of Mice with GABA_{A} Receptor Potentiator or Antagonist

To investigate the effect of GABA_{A} receptor potentiator or antagonist treatment on NSC proliferation, female mice (6-7 months of age) were treated with once-daily intraperitoneal injections of pentobarbital (PB) (Sigma, 50 mg/kg; apoE4-KI mice) or picrotoxin (PTX) (Sigma, 4 mg/kg; apoE3-KI mice). Twenty-four hours after the last treatment, mice received two injections of BrdU (100 mg/kg) 6 h apart and were sacrificed 1 day later. To investigate the effect of GABA_{A} receptor potentiator or antagonist treatment on neuronal maturation, female mice (6-7 months of age) received two injections of BrdU 6 h apart. Twenty-four hours later, apoE3-KI mice received daily injections of PTX for 7 consecutive days, and apoE4-KI mice received daily injections of PB for 28 consecutive days. Mice were sacrificed 4 weeks after the second BrdU injection.

### Stereotaxic Surgery of Engineered Retrovirus Expressing GFP

Engineered self-inactivating murine retrovirus expressing GFP was used to specifically label proliferating cells and their progeny (van Pragg et al., 2002). Virus was prepared by cotransfecting three constructs (encoding GFP, vesicular stomatitis virus-glycoprotein (VSV-G), and gag/pol) into HEK293T cells, purified by centrifugation, and stereotaxically injected into the dentate gyrus of wildtype, apoE3-KI, apoE4-KI, and apoE-KO mice. Mice were sacrificed 2 weeks after injection for electrophysiologic studies and 4 weeks after injection for studies of dendritic development.

### Immunostaining and Quantification of Neurogenesis and Dendritic Development of Newborn Neurons and GABAergic Interneurons

Mouse brains were fixed in 4% paraformaldehyde for 3 days, and coronal sections (40 μm) were cut continuously throughout the entire hippocampus with a vibratome. Every eighth section was immunostained with various primary and secondary antibodies and examined with a laser-scanning confocal system.

Immunostaining was carried out with anti-BrdU (mouse monoclonal, Chemicon, 1:400; or rat monoclonal, Abcam, 1:400) and/or with antibodies against other cellular markers, including mouse anti-NeuN (Chemicon, 1:400), mouse anti-nestin (Chemicon, 1:50), goat anti-Dcx (Santa Cruz Biotechnology, 1:40), goat anti-Sox2 (Santa Cruz Biotechnology, 1:350), rabbit anti-GFAP (Invitrogen, 1:400), mouse anti-β-III-tubulin (Promega, 1:800), mouse anti-S100β (Abcam, 1:400), and rat anti-Noggin (R&D Systems, 1:200). Corresponding secondary antibodies (Texas Red and fluorescein, 1:250, Vector Laboratories) were used. For Ki67, somatostatin, and GAD67 immunostaining, rabbit anti-Ki67 (Bethyl Labs, 1:1000), rat anti-somatostatin (Chemicon, 1:100), and mouse anti-GAD67 (Chemicon, 1:1500) were used with biotinylated goat anti-rabbit IgG (1:250), rabbit anti-rat IgG (1:250), and rabbit anti-mouse IgG (1:250) as secondary antibodies and diaminobenzadine as a chromagen (Dako).

Mouse brains were fixed in 4% paraformaldehyde for 3 days, and coronal sections (40 μm) were cut continuously throughout the entire hippocampus with a vibratome. Single- or double-immunostained cells on both sides of the hippocampus of all stained sections were counted and calculated. Numbers of newborn cells (BrdU⁺) and immature neurons (BrdU⁺ and Dcx⁺) in the SGZ were determined 1 or 3 days after BrdU injection; numbers of surviving newborn cells (BrdU⁺) and mature neurons (BrdU⁺ and NeuN⁺) in the SGZ were determined 4 or 10 weeks after injection. Newborn neurons that had migrated from the SVZ to the OB were quantified 4 weeks after BrdU injection as reported (Galvão et al., 2008). The number of GABAergic interneurons in the hilus of the dentate gyrus was determined by counting GAD67- and somatostatin-positive cells.

The dendritic structure of GFP⁺ newborn neurons was imaged with a confocal microscope, and the dendritic processes were reconstructed in three dimensions by merging Z-series stacks of 10-18 sections. All GFP⁺ neurons with largely intact dendritic trees were analyzed for total dendritic length and branch number (Duan et al., 2007; Ge et al., 2006a).

### Primary Hippocampal Neuronal Culture and Quantification of Neuronal Survival

Primary hippocampal neuronal cultures were prepared from P0 pups of homozygous apoE3-KI, apoE4-KI, wildtype, and apoE-KO mice (Chen et al., 2005). In some experiments, primary neurons were transduced with lenti-tau-shRNA viruses (Open Biosystems) at 5 days in culture to knock down tau expression. After 14 days in vitro, the cultures were fixed in 4% paraformaldehyde and immunostained for anti-MAP2 and anti-GAD67. To measure neuronal survival in hippocampal neuron cultures, MAP2- and GAD67-positive neurons were counted in 15-30 random fields under a fluorescence microscope (200× magnification) (Chen et al., 2005). In parallel experiments, human full-length apoE, p-tau, and total tau in cell lysates were analyzed by western blotting (Brecht et al., 2004; Huang et al., 2001) and quantified.

### NSC Culture and Neural Differentiation In Vitro

NSCs were isolated from brains of apoE3-KI, apoE4-KI, wildtype, and apoE-KO mice at postnatal day 1 (P1) using a modified neurosphere method (Brewer and Torricelli, 2007; Ray and Gage, 2006). The expression of apoE and Noggin in NSCs with different apoE genotypes was assessed by anti-apoE and anti-Noggin (R&D Systems) western blots of cell lysates. Neural differentiation of the established NSCs with different apoE genotypes was induced in vitro as reported (Song et al., 2002). In some experiments, recombinant mouse Noggin (R&D Systems) was added at 500 ng/ml into the medium during neural differentiation. Neuronal and astrocytic differentiation of NSCs at 7 days was determined and quantified.

### Mass Spectrometry Analysis of GABA Release

Female apoE3-KI and apoE4-KI mice (6-7 months of age) were decapitated, and the hippocampi were isolated and sliced. The slices were preincubated for 10 min at 37° C. in 200 μl of 95% O₂- and 5% CO₂-saturated basal medium with Eagle's salts (BME). The basal and depolarization-evoked GABA release were determined by matrix-assisted laser desorption/ionization mass spectrometry (Bolteus and Bordey, 2004).

### Electrophysiology

ApoE3-KI and apoE4-KI mice (2-3 months) were sacrificed 2 weeks after retrovirus-GFP injection and processed for slice preparation as described (Duan et al., 2007; Ge et al., 2006a). Whole-cell voltage-clamp recordings from visually identified GFP⁺ neurons (2 weeks after injection) were obtained with an infrared differential interference contrast video microscopy system.

### Statistical Analysis

Values are expressed as mean±SD. The statistical significance of the difference between means was assessed with unpaired, two-sample t tests. The statistical significance of the difference in total dendritic length or dendritic branch number was assessed by the Kolmogorov-Smirnov test. p<0.05 was considered statistically significant.

### Results

### Adult NSCs Express ApoE

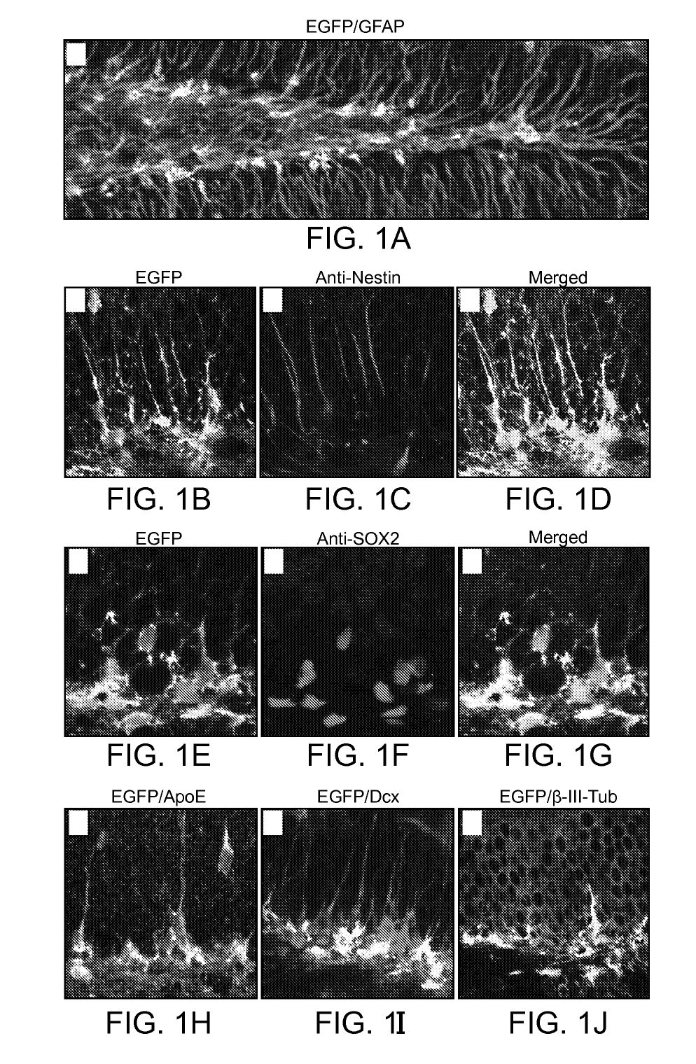

To study apoE expression in the neurogenic niches of the adult central nervous system, a previously generated EGFP_{apoE} reporter mouse was used, in which a cDNA encoding enhanced green fluorescent protein (EGFP) with a stop codon was inserted by gene targeting into the apoE gene locus immediately after the translation initiation site. In heterozygous EGFP_{apoE} reporter mice, one apoE allele was sufficient to maintain normal lipid metabolism while the EGFP reporter allele provided a real-time location marker of apoE expression in vivo (Xu et al., 2006). Confocal imaging of the dentate gyrus revealed that the neurogenic SGZ was densely populated with EGFP-positive cells (FIG. 1A). To test if EGFP expression was localized to NSCs, we immunostained brain sections with antibodies against nestin, a cytoskeletal protein expressed predominantly in the processes of NSCs, and Sox2, another NSC marker in the brain (Suh et al., 2007). The EGFP-positive cells were positive for both nestin, particularly in their radial processes (FIGS. 1B-1D), and Sox2 (FIGS. 1E-1G). The EGFP-positive cells were also positive for apoE (FIG. 1H). Confirmation was carried out by triple immunostaining of the SGZ that endogenous apoE expression was present in nestin/Sox2 double positive NSCs. Moreover, nestin/Sox2 double positive NSCs in the SVZ and rostral migratory stream (RMS) of both wildtype and EGFP_{apoE} reporter mice also expressed apoE. The EGFP-positive cells, however, were not positive for doublecortin (Dcx), expressed by immature neurons, or the ubiquitously expressed neuronal marker β-III-tubulin (FIGS. 1I and 1J). Consistent with the findings in the SGZ, EGFP expression in the apoE reporter mouse was turned off when SVZ NSCs developed into immature neurons expressing Dcx. The apoE expression in NSCs was further confirmed by western blot analysis of in vitro cultured NSCs from brains of wildtype, apoE3-KI, and apoE4-KI mice (it is noted that mouse apoE is 5 amino acids shorter than human apoE). The strong and specific expression of apoE in NSCs suggested that apoE may play a role in adult neurogenesis.

FIGS. 1A-J. ApoE Is Expressed in Hippocampal NSCs (A-J) Confocal images of the dentate gyrus of EGFP_{apoE} reporter mice. Green indicates EGFP representing apoE (A, B, D, E, G-J). Red indicates immunostaining positive for anti-GFAP (A), anti-nestin (C, D), anti-Sox2 (F, G), anti-apoE (H), anti-Dcx (I), or anti-β-III-tubulin (J).

### Neurogenesis is Reduced but Astrogenesis Increased in Apoe-KO Mice

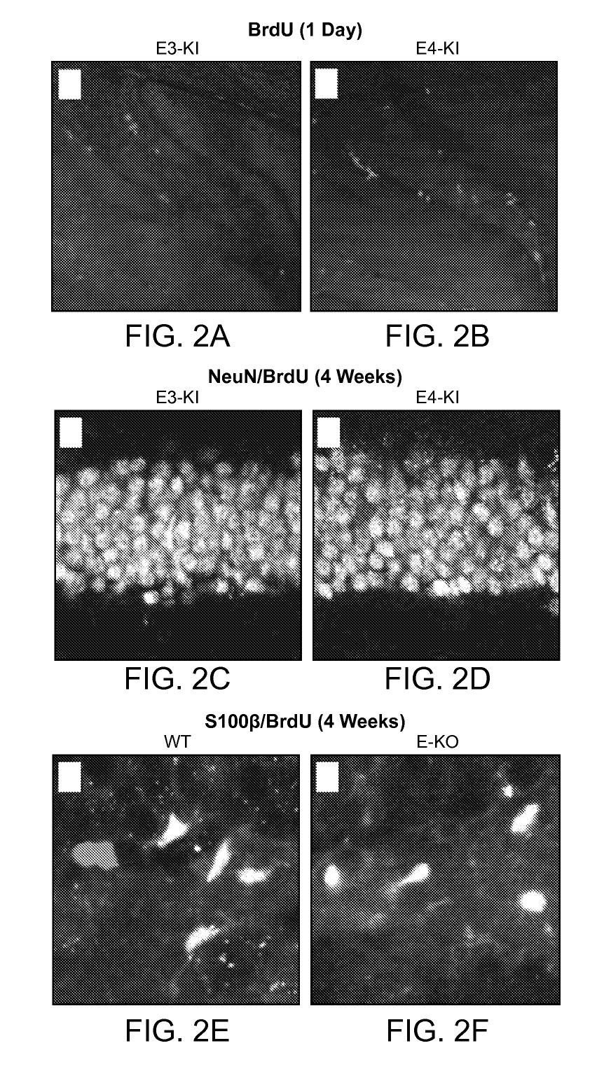

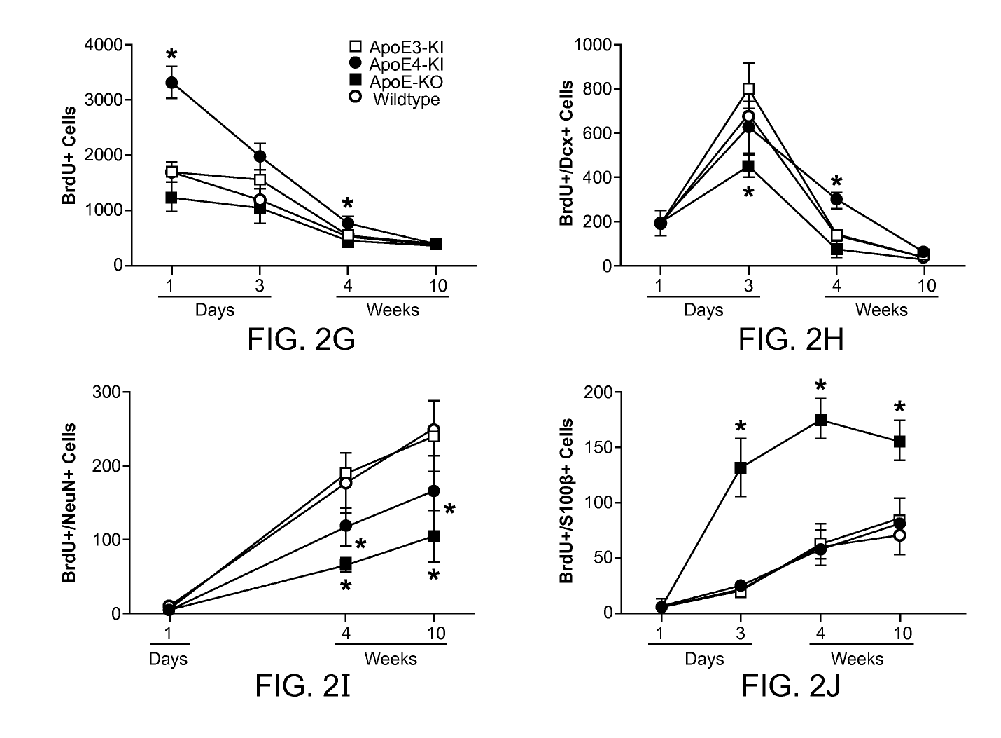

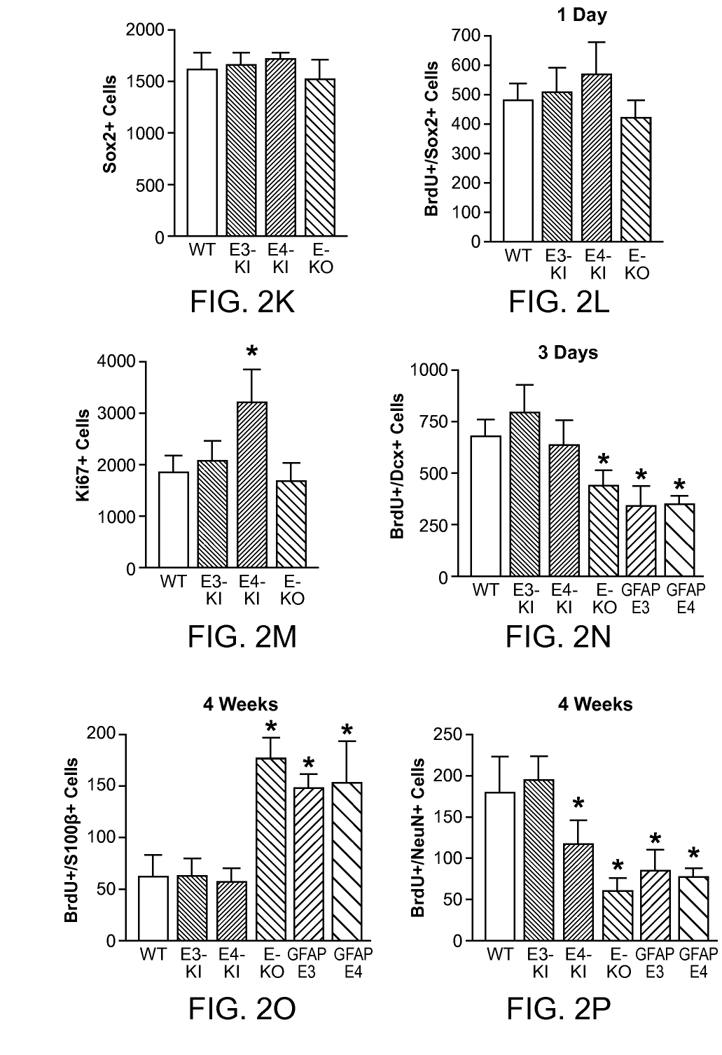

To analyze hippocampal neurogenesis and astrogenesis as a consequence of apoE genotype, survival and differentiation of newborn cells were followed over time in 6-7-month-old mice with knockout for apoE (apoE-KO) or with knock-in alleles for human apoE3 or apoE4 (apoE3-KI or apoE4-KI). At early time points after intraperitoneal injection of 5-bromo-2′-deoxyuridine (BrdU), numbers of newborn cells were comparable between apoE-KO and wildtype mice (FIG. 2G); however, co-labeling of BrdU positive cells with Dcx, NeuN, or the astrocytic marker S100β indicated that the newly generated cells predominantly differentiated into astrocytes in apoE-KO mice (FIGS. 2E, 2F, 2H, 2I, and 2J). The increase of astrogenesis in apoE-KO mice was confirmed by the significantly greater number of astrocytes (S100β⁺) in the hippocampal hilus of apoE-KO mice compared with wildtype mice.

ApoE deficiency did not significantly affect the number of newborn cells (BrdU⁺) (FIG. 2G) or Sox2-positive cells (FIG. 2K). The latter cells represent total NSCs. Nor did apoE deficiency affect the number of BrdU and Sox2 double-positive cells (FIG. 2L), which reflect self-renewal of NSCs, in the SGZ. Taken together, these results suggest that maintaining apoE function and/or its mediated lipid metabolism is required to ensure proper neuronal differentiation of NSCs.

Increased BMP Signaling Mediates the Imbalance between Astrogenesis and Neurogenesis in ApoE-KO Mice

The bone-morphogenetic protein (BMP) inhibitor Noggin is known to inhibit astrogenesis and stimulate neurogenesis (Lim et al., 2000) and its expression in murine SGZ and SVZ was previously reported (Fan et al., 2003; Lim et al., 2000; Tang et al., 2009). It was found that Sox2/apoE double positive NSCs in the SGZ and SVZ also expressed Noggin. Interestingly, NSCs cultured from apoE-KO mice had ˜80% lower Noggin protein levels, as determined by anti-Noggin western blot, compared with NSCs cultured from wildtype mice. Addition of recombinant mouse Noggin to the culture of NSCs from apoE-KO mice under conditions of neuronal differentiation inhibited astrogenesis and stimulated neurogenesis to levels similar to those of NSCs from wildtype mice. Thus, increased BMP signaling in apoE deficient NSCs appears to promote glial differentiation at the expense of neurogenesis.

NSC Proliferation is Increased but Neuronal Maturation Decreased in ApoE4-KI Mice Concomitant with Reduced Neurogenesis

Proliferation and differentiation of NSCs in the SGZ over time in apoE3-KI mice was virtually indistinguishable from wildtype mice, suggesting that human apoE3 is functionally equivalent to mouse apoE in supporting hippocampal neurogenesis, including NSC proliferation (FIG. 2G), generation of immature neurons (FIG. 2H), mature neurons (FIG. 2I), and astrocytes (FIGS. 2J). In contrast, apoE4-KI mice had twofold more BrdU-positive cells in the SGZ than apoE3-KI mice one day after BrdU injection (FIGS. 2A, 2B, and 2G). Similarly, Ki67 immunostaining showed that apoE4-KI mice had ˜60% more proliferating cells than apoE3-KI, wildtype, and apoE-KO mice (FIG. 2M). However, apoE4 affected neither the total number of Sox2-positive cells (FIG. 2K) nor the number of BrdU/Sox2 double-positive cells (FIG. 2L) in the SGZ. Thus, although proliferation is increased in the SGZ of apoE4-KI mice, the number of NSCs and their self-renewal are normal.

At 4 weeks after BrdU injection, apoE4-KI mice had ˜50% fewer mature neurons in the SGZ than apoE3-KI and wildtype mice (FIGS. 2C, 2D, and 2I) but threefold more immature neurons (FIG. 2H) and similar numbers of astrocytes (FIG. 2J). These results suggest that apoE4 affects the maturation of newborn neurons in the hippocampus but does not have an effect on astrogenesis. At 10 weeks after injection, apoE4-KI mice still had significantly fewer mature neurons (FIG. 2I), although the number of immature neurons had decreased nearly to baseline levels in both groups (FIG. 2H). Furthermore, at 4 weeks after BrdU injection, apoE4-KI mice also had ˜25% fewer mature neurons in the olfactory bulb (OB), which originated from the SVZ, than apoE3-KI mice. Thus, neurogenesis is reduced in both the SGZ and the SVZ/OB of apoE4-KI mice, with a more pronounced effect in the SGZ.

### Effect of Apoe Deficiency on Hippocampal Neurogenesis is Cell-Autonomous but Effect of ApoE4 is Non-Cell-Autonomous

To determine whether apoE deficiency has a cell-autonomous effect on hippocampal neurogenesis, GFAP-apoE3 and GFAP-apoE4 transgenic mice on a mouse apoE-KO background were studied. In these mice, human apoE is expressed only in adult astrocytes and is secreted into the intercellular space in the brain (Brecht et al., 2004). Immunofluorescence staining revealed that nestin-positive NSCs in the SGZ and SVZ did not express apoE in GFAP-apoE4 and GFAP-apoE3 transgenic mice, although mature astrocytes in the hippocampus or other brain regions did express apoE. At 3 days after BrdU injection, GFAP-apoE3, GFAP-apoE4, and apoE-KO mice had similar numbers of newly generated immature neurons (BrdU⁺/Dcx⁺) but significantly fewer than wildtype, apoE3-KI, and apoE4-KI mice (FIG. 2N), suggesting that astrocyte-secreted apoE does not support neuronal differentiation of NSCs. At 4 weeks, GFAP-apoE3, GFAP-apoE4, and apoE-KO mice had similar numbers of newly generated astrocytes (BrdU⁺/S100β⁺) but significantly more than wildtype, apoE3-KI, and apoE4-KI mice (FIG. 2O), suggesting that astrocyte-secreted apoE does not suppress astrocytic differentiation of NSCs. GFAP-apoE3, GFAP-apoE4, apoE-KO, and apoE4-KI mice also had similar numbers of mature neurons (BrdU⁺/NeuN⁺) but significantly fewer than wildtype and apoE3-KI mice (FIG. 2P). Thus, NSC-expressed apoE is required to support hippocampal neurogenesis and suppress astrogenesis.

To further test the effects of apoE deficiency and apoE4 on neurogenesis, neural differentiation of cultured NSCs in vitro was analyzed. At 7 days in culture, double immunostaining for MAP2 and GFAP revealed a significantly lower percentage of neurons, but a much higher percentage of astrocytes, generated from NSCs of apoE-KO mice than from those of wildtype mice. The percentages of neurons and astrocytes generated from NSCs of apoE3-KI, apoE4-KI, and wildtype mice were similar. Thus, the effect of apoE deficiency on hippocampal neurogenesis from NSCs is cell-autonomous but the effect of apoE4 is non-cell-autonomous.

FIGS. 2A-P. Hippocampal Neurogenesis and Astrogenesis in Mice with Knockout for ApoE or with Knock-in Alleles for Human ApoE3 or ApoE4 (A, B) Representative confocal images of the BrdU-positive cells in the SGZ of female apoE3-KI (A) and apoE4-KI (B) mice at 6-7 months of age were collected 1 day after BrdU injection. (C, D) Representative confocal images of the BrdU and NeuN double positive cells in the SGZ of female apoE3-KI (C) and apoE4-KI (D) mice at 6-7 months of age were collected 4 weeks after BrdU injection. (E, F) Representative confocal images of the BrdU and S100β double positive cells in the SGZ of female wildtype (E) and apoE-KO (F) mice at 6-7 months of age were collected 4 weeks after BrdU injection. (G-J) Numbers of newborn cells (BrdU⁺) (G), immature neurons (BrdU⁺/Dcx⁺) (H), mature neurons (BrdU⁺/NeuN⁺) (I), and astrocytes (BrdU⁺/S100β⁺) (J) in the SGZ of female mice of various apoE genotypes at 6-7 months of age were determined 1 and 3 days and 4 and 10 weeks after BrdU injection. Values are mean±SD (n=4-6 mice per genotype). *p<0.05 versus other groups (t test). (K) Total numbers of Sox2-positive cells in the SGZ of female wildtype, apoE3-KI, apoE4-KI, and apoE-KO mice at 6-7 months of age. Values are mean±SD (n=4 mice per genotype). (L) Numbers of BrdU and Sox2 double-positive cells in the SGZ of female wildtype, apoE3-KI, apoE4-KI, and apoE-KO mice at 6-7 months of age were determined 1 day after BrdU injection. Values are mean±SD (n=4 mice per genotype). (M) Total numbers of Ki67-positive cells in the SGZ of female wildtype, apoE3-KI, apoE4-KI, and apoE-KO mice at 6-7 months of age. Values are mean±SD (n=4 mice per genotype). *p<0.05 versus other groups. (N-P) Numbers of immature neurons (BrdU⁺/Dcx⁺) (N), astrocytes (BrdU⁺/S100β⁺) (O), and mature neurons (BrdU⁺/NeuN⁺) (P) in the SGZ of female mice with various apoE genotypes at 6-7 months of age were determined at 3 days and 4 weeks after BrdU injection. Values are mean±SD (n=4-6 mice per genotype). *p<0.05 versus wildtype and apoE3-KI mice (t test).

### Dendritic Development of Newborn Hippocampal Neurons is Impaired in ApoE4-KI Mice

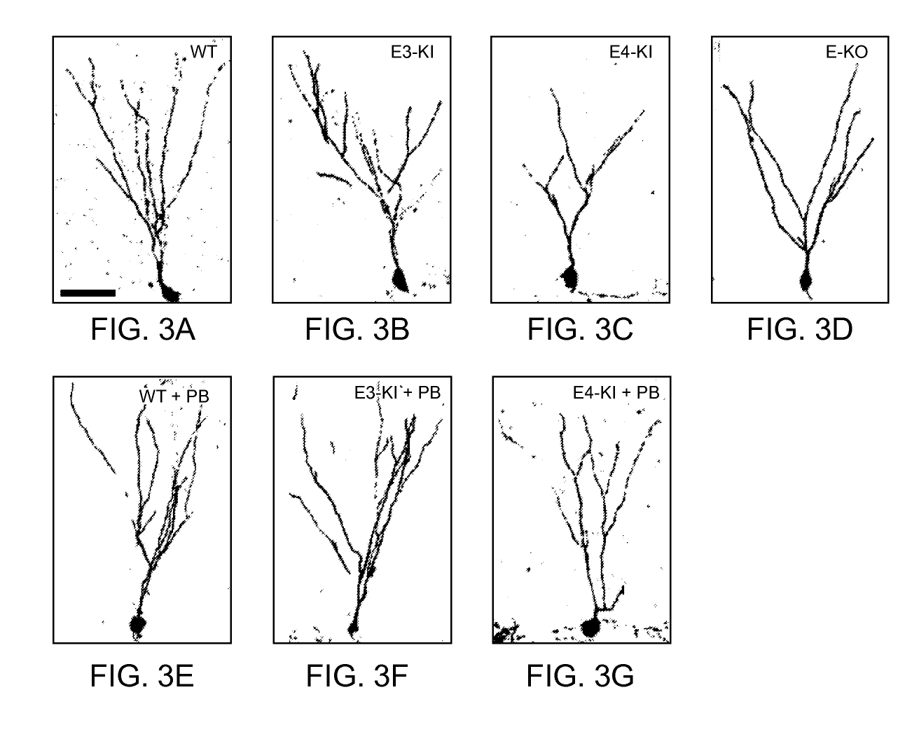

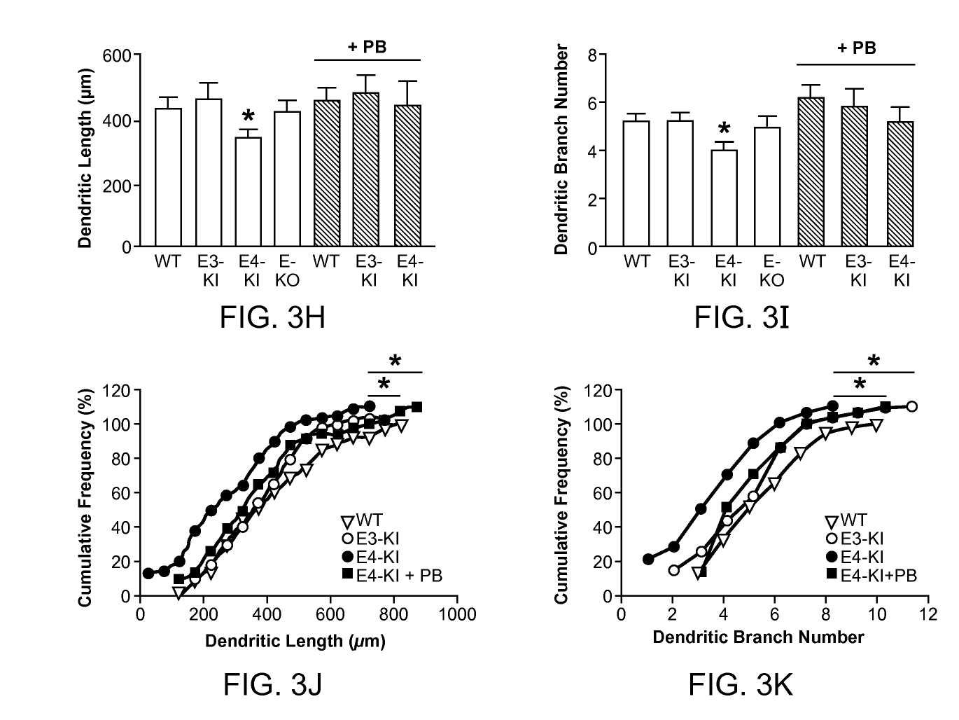

The time course studies of proliferation and differentiation of NSCs suggested that neuronal maturation was delayed or impaired in apoE4-KI mice, possibly explaining the impaired neurogenesis. To determine if the apoE4 genotype has an effect on dendritic development, the dendritic arbors of newborn hippocampal neurons were reconstructed from confocal microscopic images. Newborn neurons were labeled by stereotaxically injecting retrovirus expressing GFP into the dentate gyrus (Ge et al., 2006a; Zhao et al., 2006). Four weeks after injection, when the newborn neurons were fully developed and integrated (Aimone et al., 2006; Ge et al., 2006a; Lie et al., 2004; Ming and Song, 2005), GFP⁺ neurons were found to have much less elaborate dendrites in apoE4-KI mice than in apoE3-KI, wildtype, and apoE-KO mice (FIGS. 3A-3D). The total dendritic length and branch number of newborn neurons were significantly lower in apoE4-KI mice (FIGS. 3H-3K).

FIGS. 3A-K. Dendritic Development of Newborn Neurons in the Hippocampus Is Reduced in ApoE4-KI Mice (A-G) Confocal three-dimensional reconstruction of dendrites (inverted images) of newborn neurons (4 weeks after retrovirus-GFP injection) in the dentate gyrus of wildtype (A), apoE3-KI (B), apoE4-KI (C), and apoE-KO (D) mice, wildtype mice treated with PB (E), apoE3-KI mice treated with PB (F), and apoE4-KI mice treated with PB (G). Scale bar, 50 μm. (H-K) Total dendritic length (H, J) and dendritic branch number (I, K) of newborn neurons. *p<0.05 (t test in H and I; Kolmogorov-Smirnov test in J and K). WT, n=43; E3-KI, n=84; E4-KI, n=73; E-KO, n=35; WT+PB, n=52; E3-KI+PB, n=42; E4-KI+PB, n=31. Values in panels H and I are mean±SEM.

### Numbers of GABAergic Interneurons in the Dentate Gyrus Are Reduced in ApoE4-KI Mice

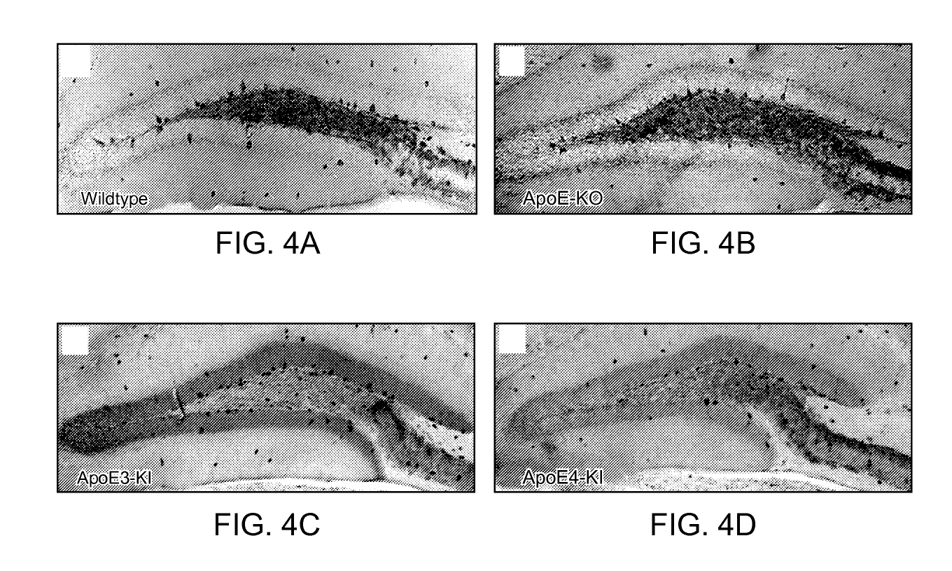

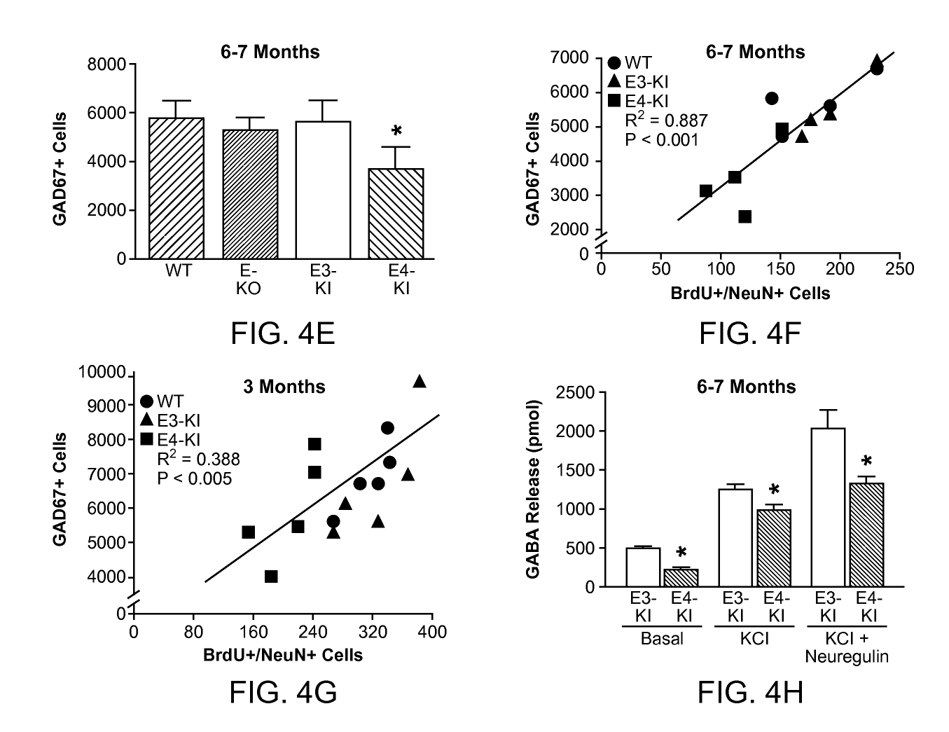

The phenotype of abnormal hippocampal neurogenesis in apoE4-KI mice—increased NSC proliferation and impaired neuronal maturation and dendritic development—mirrors that in mice with GABA signaling inhibition (Earnheart et al., 2007; Ge et al., 2006a; Ge et al., 2006b; Liu et al., 2006; Tozuka et al., 2005). To determine whether apoE4 impairs GABAergic interneurons in the hilus of the hippocampus, anti-GAD67 immunostaining was performed for GABAergic interneurons in wildtype, apoE-KO, apoE3-KI, and apoE4-KI mice (FIGS. 4A-4D). At 6-7 months of age, apoE4-KI mice had ˜30% fewer GAD67-positive interneurons in the hilus than wildtype, apoE-KO, and apoE3-KI mice (FIG. 4E). Importantly, the number of GAD67-positive GABAergic interneurons correlated positively with the number of newly generated mature neurons (BrdU⁺/NeuN⁺) in the SGZ of wildtype, apoE3-KI, and apoE4-KI mice (FIG. 4F). A similar positive correlation was also observed in these groups of mice at 3 months of age (FIG. 4G). Similar results were obtained by anti-somatostatin immunostaining for GABAergic interneurons in mice at 6 months of age. Thus, the reduced neuronal maturation observed in the SGZ of apoE4-KI mice could be the result of reduced innervation from GABAergic interneurons in the hilus. Consistent with the finding of GABAergic interneuron reduction, both basal and KCl− or neuregulin-evoked GABA release in hippocampal slices were significantly lower in apoE4-KI than apoE3-KI mice (FIG. 4H), as determined by mass spectrometry. Furthermore, the axonal termini of GABAergic interneurons on granule cells in the dentate gyrus were also significantly decreased at both the absolute level and relative to the presynaptic marker synaptophysin. Interestingly, apoE3-KI, wildtype, and apoE-KO mice had similar numbers of GABAergic interneurons (FIG. 4E) and axonal termini onto dentate gyrus granule cells, suggesting that apoE deficiency does not decrease the number of GABAergic interneurons or their axonal termini.

At 12-13 months of age, apoE4-KI mice had ˜40% fewer GAD67-positive interneurons in the hilus than apoE3-KI mice. Newly generated mature neurons in the SGZ similarly decreased in apoE4-KI mice. However, at 1 month of age, apoE4-KI, apoE3-KI, wildtype, and apoE-KO mice had similar numbers of GAD67-positive GABAergic interneurons in the hilus (7527±593, 8320±804, 7740±1751, 7256±1545, n=4, p>0.05), suggesting that the effect of apoE4 on GABAergic interneurons is not due to an early developmental impairment but occurs during adult neurogenesis.

FIGS. 4A-H. Numbers of GABAergic Interneurons and GABA Release in the Hippocampus of ApoE4-KI Mice Are Reduced (A-D) Immunostaining of GAD67-positive GABAergic interneurons in the hilus of female wildtype (A), apoE-KO (B), apoE3-KI (C), and apoE4-KI (D) mice at 6-7 months of age. (E) Numbers of GAD67-positive GABAergic interneurons in different mice at 6-7 months of age. Values are mean±SD (n=4-7 mice per genotype). *p<0.05 versus other groups of mice (t test). (F, G) Positive correlation between the number of GAD67-positive interneurons and the number of BrdU⁺/NeuN⁺ neurons among female wildtype, apoE3-KI, and apoE4-KI mice at 6-7 months of age (F, n=12 mice) and at 3 months of age (G, n=15 mice). (H) GABA release in hippocampal slices, determined by mass spectrometry. Values are mean±SD (n=4-7 mice per genotype). *p<0.05 versus apoE3-KI mice (t test).

GABAergic Interneuron Survival is Decreased in Primary Hippocampal Neuronal Cultures from ApoE4-KI Mice, Concomitant with Increased Tau Phosphorylation and Generation of Neurotoxic ApoE Fragments

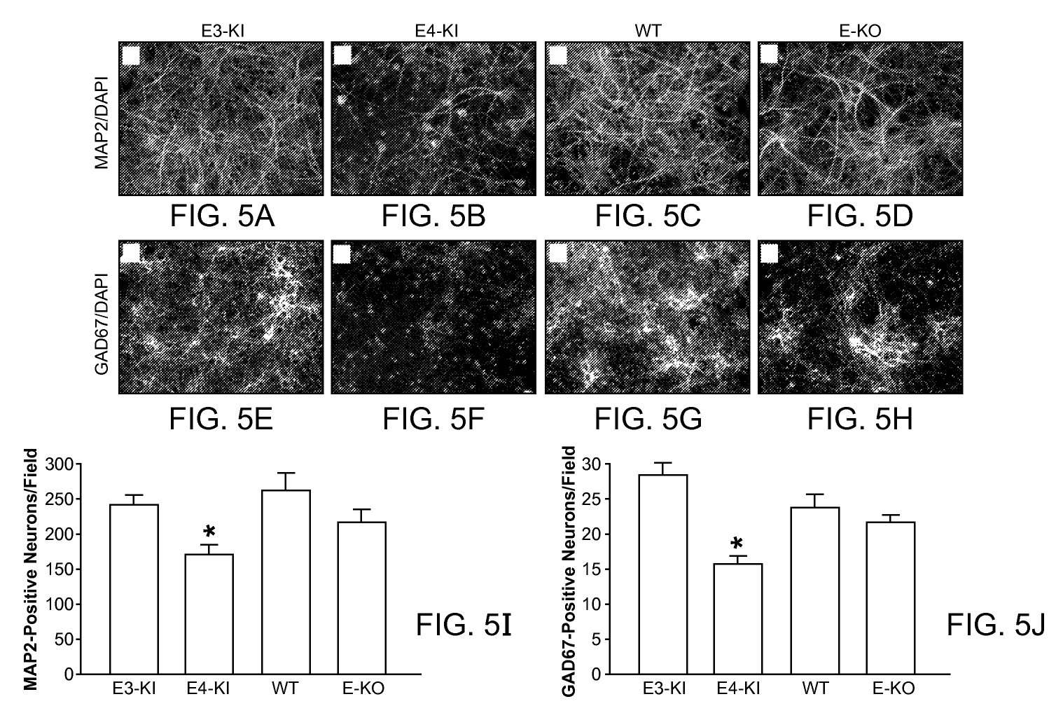

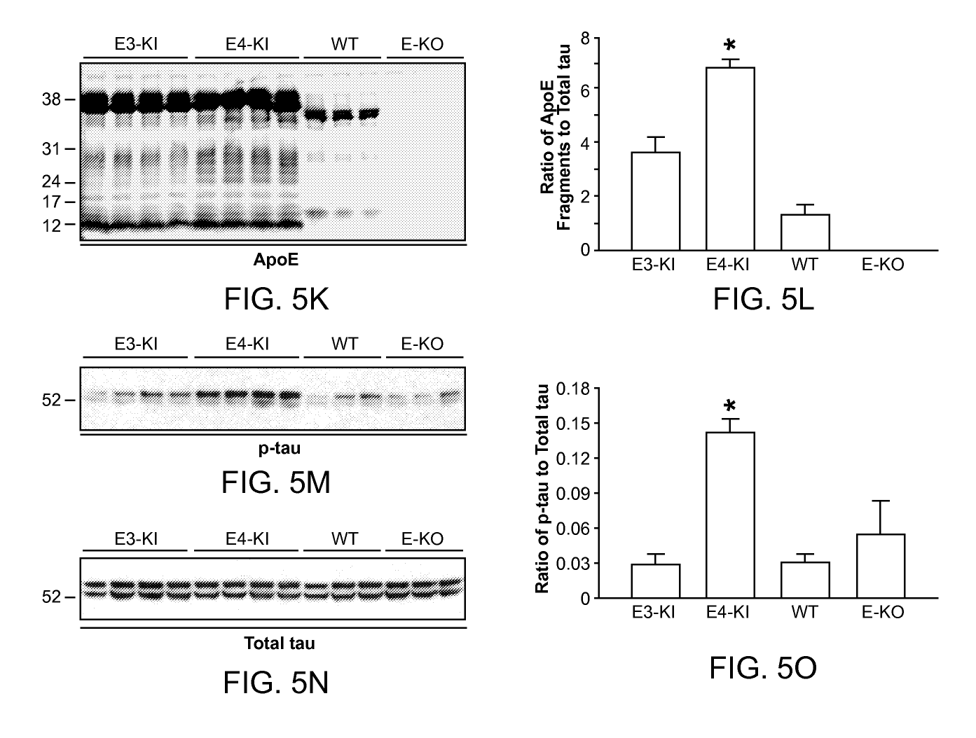

To determine the mechanisms of the detrimental effects of apoE4 on GABAergic interneurons, primary hippocampal neurons from apoE3-KI, apoE4-KI, wildtype, and apoE-KO mice were analyzed. After 14 days of in vitro culture, immunostaining for MAP2 (a neuronal marker) and GAD67 (a GABAergic neuronal marker) revealed ˜25% and ˜45% lower survival of total and GABAergic neurons, respectively, from apoE4-KI mice than from apoE3-KI mice (FIGS. 5A-5J). It was reported previously that neurons under stress, including neurons cultured in vitro (Harris et al., 2004; Xu et al., 2008), express apoE and that neuronal apoE undergoes proteolytic cleavage to generate neurotoxic fragments in vitro and in vivo, with apoE4 being more susceptible to the cleavage than apoE3 (Brecht et al., 2004; Chang et al., 2005; Harris et al., 2003; Huang et al., 2001). In the current study, significantly more apoE fragments were generated in neurons from apoE4-KI mice than in those from apoE3-KI and wildtype mice, as shown by western blot with anti-apoE (FIGS. 5K and 5L). The apoE4 fragmentation pattern was very similar to that in the brains of neuron-specific apoE4 transgenic mice and humans with AD (Brecht et al., 2004; Harris et al., 2003; Huang et al., 2001).

Since apoE4 fragments generated in neurons can increase tau phosphorylation, which is one of the major pathological hallmarks of AD (Tanzi and Bertram, 2001), leading to neuronal cell death in vitro and in vivo (Brecht et al., 2004; Chang et al., 2005; Harris et al., 2003; Huang et al., 2001), the levels of phosphorylated tau (p-tau) in neurons from mice with different apoE genotypes were determined. Clearly, neurons from apoE4-KI mice had significantly higher levels of p-tau than those from apoE3-KI, wildtype, and apoE-KO mice, as shown by western blot with anti-p-tau (FIGS. 5M-5O). Anti-GAD67 and anti-p-tau double immunostaining revealed ˜fourfold more p-tau positive GABAergic neurons from apoE4-KI mice than from apoE3-KI mice, although the total numbers of GABAergic neurons were ˜45% less from apoE4-KI mice than from apoE3-KI mice (FIGS. 5A-5J). Thus, over 70% of GABAergic neurons from apoE4-KI mice were positive for p-tau, as compared to ˜10% of them from apoE3-KI mice.

To further determine the relationship of apoE4-induced tau pathology and GABAergic neuron death, we knocked down tau (˜70%) in primary hippocampal neurons from apoE3-KI and apoE4-KI mice using a lentiviral tau-shRNA approach. Knocking down tau significantly increased the survival of total and GABAergic neurons from apoE4-KI mice, reaching levels similar to those of neurons from apoE3-KI mice. Knocking down tau did not significantly alter the survival of total and GABAergic neurons from apoE3-KI mice. Thus, apoE4 impairs the survival of GABAergic interneurons by generating more neurotoxic apoE fragments and increasing p-tau levels, leading to GABAergic interneuron death, which can be fully rescued by lowering the endogenous tau level.

FIGS. 5A-O. Levels of Neurotoxic ApoE Fragments and Tau Phosphorylation Are Increased, and GABAergic Neuron Survival Is Decreased in Primary Hippocampal Neuronal Cultures from ApoE4-KI mice (A-H) Primary hippocampal neuron cultures were prepared from P0 pups of apoE3-KI, apoE4-KI, wildtype, and apoE-KO mice, cultured for 14 days in vitro (14 DIV), and stained with anti-MAP2 (red) and DAPI (blue) (A-D) or anti-GAD67 (green) and DAPI (blue) (E-H). Shown are representative images from five coverslips of each genotype and five fields per coverslip (magnification, 200×). (I, J) MAP2-positive (I) and GAD67-positive (J) neurons were quantified. Values are mean±SEM (five images per coverslip and five coverslips per genotype). *p<0.05 versus other groups (t test). (K) Anti-apoE western blot of primary neuron lysates from apoE3-KI, apoE4-KI, wildtype, and apoE-KO mice. Note that mouse apoE is 5 amino acids shorter than human apoE. (L) ApoE fragmentation, reported as the ratio of total apoE fragments to total tau. Values are mean±SD (n=3-4 mice per genotype). *p<0.001 versus other groups (t test). (M, N) Anti-p-tau (M, AT8 monoclonal antibody) and anti-total tau (N, tau-5 monoclonal antibody) western blots of primary neuron lysates from apoE3-KI, apoE4-KI, wildtype, and apoE-KO mice. (O) The level of tau phosphorylation, reported as the ratio of p-tau to total tau. Values are mean±SD (n=3-4 mice per genotype). *p<0.001 versus other groups (t test).

### Presynaptic GABAergic Input to Newly Born Neurons is Reduced in ApoE4-KI Mice

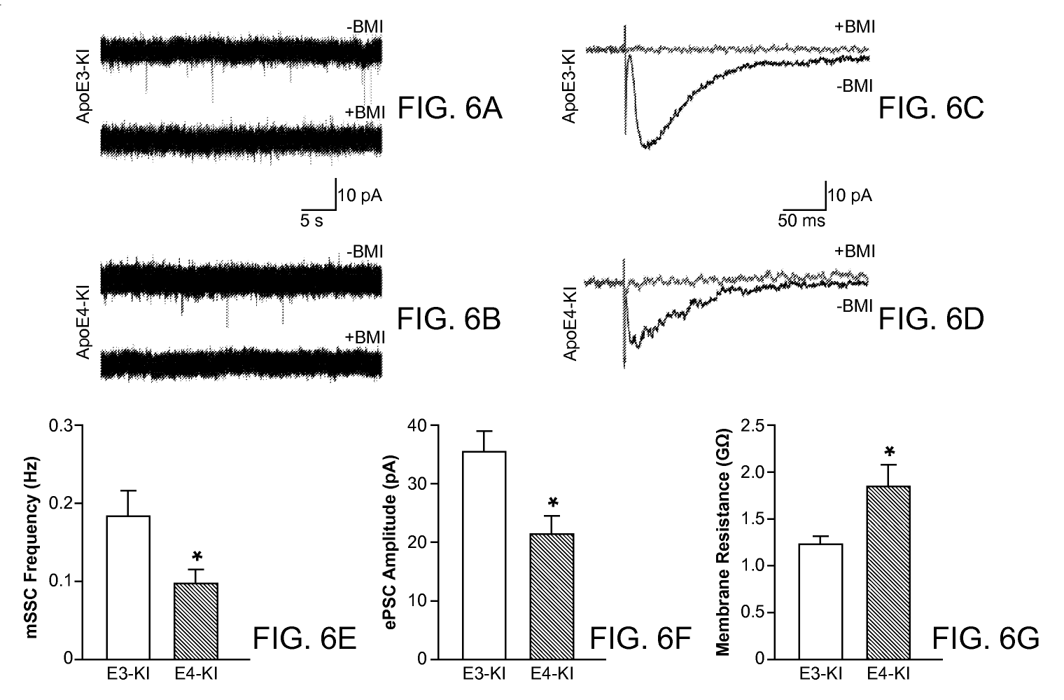

To assess the functional consequence of the decreased number of GABAergic interneurons in the hilus, whole-cell patch-clamp recordings from newborn granule cells in acute slices of hippocampus from retrovirus-GFP-injected apoE3-KI and apoE4-KI mice were performed. Two weeks after stereotaxical viral injection, when GABAergic input is critical for neuronal maturation of newborn cells (Ge et al., 2006a; Ge et al., 2006b; Liu et al., 2006; Tozuka et al., 2005), about 90% of GFP⁺ newborn neurons in both groups had active GABAergic spontaneous synaptic currents (SSCs). However, GFP⁺ neurons in apoE4-KI mice had significantly higher input resistance (FIG. 6G). Since the input resistance of newborn neurons decreases as they mature (Duan et al., 2007; Espósito et al., 2005), these results suggest a delayed maturation of newborn neurons in apoE4-KI mice.

Next the level of GABAergic synaptic innervation, which is critical for neuronal development of newborn hippocampal cells (Ge et al., 2006a; Ge et al., 2006b; Liu et al., 2006; Tozuka et al., 2005), was assessed. GABAergic miniature SSCs (mSSCs) were recorded from GFP⁺ neurons (resting membrane potential Vₘ=−65 mV) 2 weeks after injection of the retrovirus-GFP construct. These studies were conducted in the presence of 6,7-dinitroquinoxaline-2,3-dione (DNQX) (20 μM) and D-(−)-2-amino-5-phosphonovaleric acid (D-AP5) (50 μM) to block glutamate-mediated currents and tetrodotoxin (TTX) (1 μM) to block action potential-mediated GABA release. Bicuculline methoiodide (BMI, 100 μM) abolished the mSSCs, confirming the contribution of GABA_{A} receptors to mSSCs (FIGS. 6A and 6B). mSSCs were detected in almost 90% of GFP⁺ neurons in apoE3-KI and apoE4-KI mice, and the mean amplitudes of mSSCs were almost identical; however, the mSSC frequency was ˜50% lower in apoE4-KI mice (FIG. 6E), consistent with the ˜50% decrease in basal GABA release in apoE4-KI mice (FIG. 4H), suggesting a significant reduction of functional presynaptic GABAergic inputs on newborn neurons in apoE4-KI mice.

In the presence of DNQX and D-AP5, GABA-evoked postsynaptic currents (ePSCs) recorded from GFP⁺ neurons were generated by electrical stimulation of GABAergic axons in the molecular layer of the dentate gyrus. This indicates a functional coupling between hilar GABAergic interneurons and newborn neurons in both apoE3-KI and apoE4-KI mice. However, the ePSC peak amplitude was ˜40% lower in apoE4-KI mice (FIGS. 6C, 6D, and 6F). This finding is consistent with a reduction of presynaptic GABAergic inputs onto newborn neurons available for electrical activation in apoE4-KI mice. However, no significant difference in the 10-90% rise time or the ePSC decay time was observed, suggesting that the GABA_{A} receptor composition and number are not significantly altered in newborn neurons in either group. Indeed, focal application of exogenous GABA (500 μM), which bypasses presynaptic elements and allows assessment of postsynaptic GABA_{A} receptor function, evoked similar current amplitudes in GFP⁺ neurons from apoE3-KI and apoE4-KI mice. This finding reconfirms the intact GABA_{A} receptor function in newborn neurons of apoE4-KI mice. These results suggest that apoE4 impairs the maturation of newborn neurons by causing GABAergic interneuron loss, which leads to impaired GABA signaling on newborn neurons.

FIGS. 6A-G. GABAergic Electrophysiological Inputs to Newborn Neurons Are Impaired in the Hippocampus of ApoE4-KI Mice (A, B) Sample traces of mSSCs in a GFP⁺ neuron 2 weeks after retrovirus-GFP injection from an apoE3-KI (A) or an apoE4-KI (B) mouse during whole-cell voltage clamp recording in the presence of DNQX (20 μM), D-AP5 (50 μM), and TTX (1 μM). The mSSCs were blocked by bath application of BMI (100 μM). Scale bars, 10 pA and 5 s. (C, D) Sample traces of ePSCs in a GFP⁺ neuron at 2 weeks after retrovirus-GFP injection from an apoE3-KI (C) or an apoE4-KI (D) mouse during whole-cell voltage clamp recording in the presence of DNQX (20 μM) and D-AP5 (50 μM). Currents were blocked by bath application of BMI (100 μM). Scale bars: 10 pA and 50 ms. (E) Average mSSC frequency in GFP⁺ neurons was lower in apoE4-KI mice than in apoE3-KI mice. Values are mean±SD (n=21-28 cells per genotype). *p<0.05 versus apoE3-KI mice (t test). (F) Average ePSC amplitude in GFP⁺ neurons was lower in apoE4-KI mice than in apoE3-KI mice. Values are mean±SD (n=21-28 cells per genotype). *p<0.05 versus apoE3-KI mice (t test). (G) Average membrane resistance of GFP⁺ neurons in apoE3-KI and apoE4-KI mice 2 weeks after retrovirus-GFP injection. Values are mean±SD (n=40 cells per genotype). p<0.005 versus apoE3-KI mice (t test).

Blocking GABA Signaling Reduces Hippocampal Neurogenesis in ApoE3-KI Mice while Potentiating GABA Signaling in ApoE4-KI Mice Restores Neurogenesis

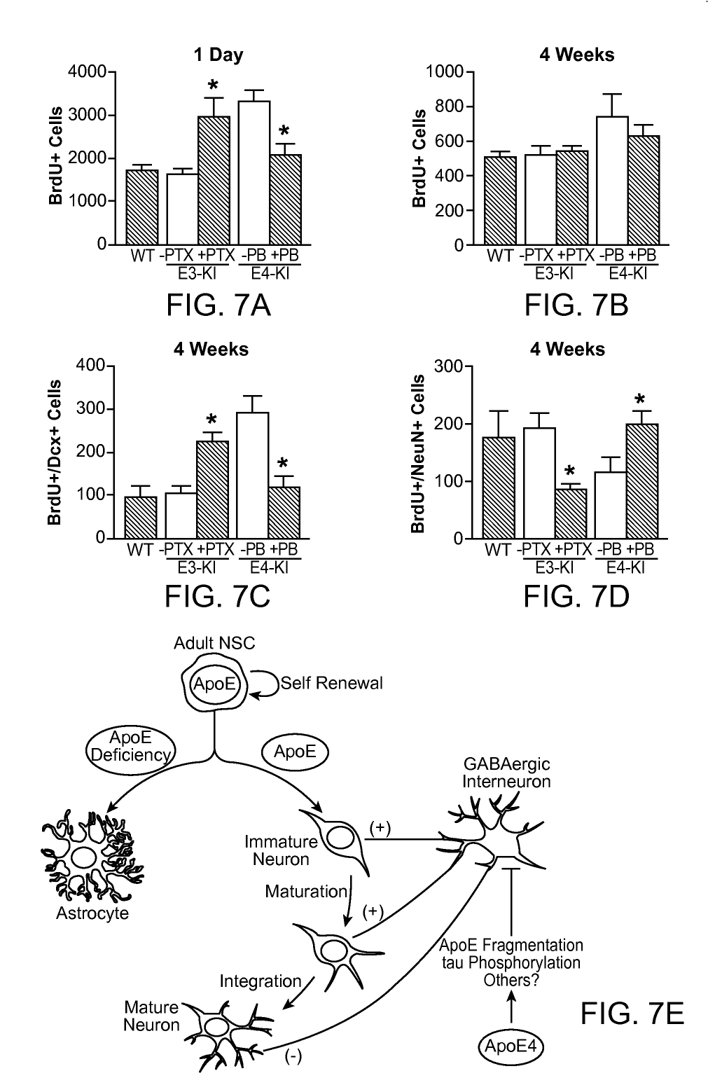

Treatment of apoE3-KI mice for 3 days with picrotoxin (PTX), a GABA_{A} receptor antagonist (Tozuka et al., 2005), increased the number of newborn cells 1 day after BrdU injection to a level similar to that in untreated apoE4-KI mice (FIG. 7A). Conversely, treatment of apoE4-KI mice with pentobarbital (PB), a GABA_{A} receptor potentiator (Tozuka et al., 2005), decreased the number of newborn cells to levels similar to those of wildtype and untreated apoE3-KI mice (FIG. 7A). Thus, the increased NSC proliferation in apoE4-KI mice likely reflects impaired GABA signaling (Liu et al., 2005; Owens and Kriegstein, 2002; Tozuka et al., 2005).

Next, apoE3-KI mice that had received BrdU injections were treated with daily injections of PTX for 7 days to inhibit GABA signaling. At 4 weeks after BrdU injection, the number of immature neurons was increased (FIG. 7C) and the number of mature neurons was decreased (FIG. 7D) in the hippocampus of apoE3-KI mice to levels similar to those in untreated apoE4-KI mice; the survival of newborn cells was unaltered (FIG. 7B). Conversely, stimulation of GABA signaling with daily injections of PB for 4 weeks decreased the number of immature neurons and increased the number of mature neurons in apoE4-KI mice, to levels similar to those in wildtype and untreated apoE3-KI mice (FIGS. 7C and 7D). Importantly, this stimulation also improved the dendritic development of newborn neurons of apoE4-KI mice, as reflected by increases in both dendritic length and branch number to levels similar to those in wildtype and apoE3-KI mice (FIGS. 3G-3K). These results further support a non-cell-autonomous effect of apoE4 on hippocampal neurogenesis from NSCs. Interestingly, stimulation of GABA signaling with PB in wildtype mice also showed a trend toward significant increase (p=0.053) in the number of dendritic branches of newborn neurons (FIGS. 3E and 3I) although the same treatment did not alter the length or number of dendritic branches of newborn neurons in apoE3-KI mice (FIGS. 3F, 3H, and 3I). Thus, treatment with a GABA_{A} receptor potentiator rescues the impairment of hippocampal neurogenesis associated with GABAergic interneuron dysfunction due to apoE4.

FIGS. 7A-E. GABA_{A} Receptor Potentiator Restores Hippocampal Neurogenesis in ApoE4-KI Mice—A Working Model for the Roles of ApoE and Its Isoforms in Adult Hippocampal Neurogenesis. Female apoE3-KI and apoE4-KI mice at 6-7 months of age were treated with a GABA_{A} receptor potentiator (PB, 50 mg/kg) or antagonist (PTX, 4 mg/kg) as described in the text. BrdU-positive cells in the SGZ were counted at 1 day (A) and 4 weeks (B), and immature neurons (C) and mature neurons (D) were counted at 4 weeks. Untreated wildtype mice at 6-7 months of age served as controls. Values are mean±SD (n=4-6 mice per genotype). *p<0.01 versus untreated mice of the same apoE genotype (t test). (E) A working model for the roles of apoE and its isoforms in adult hippocampal neurogenesis. Adult hippocampal NSCs express apoE, which plays an important role in cell fate determination of NSCs toward neuronal development. ApoE deficiency stimulates astrogenesis and inhibits neurogenesis. ApoE4 decreases hippocampal neurogenesis by inhibiting neuronal maturation of NSCs through impairing presynaptic GABAergic input onto newborn neurons.

### REFERENCES

Aimone, J. B., Wiles, J., and Gage, F. H. (2006). Potential role for adult neurogenesis in the encoding of time in new memories. Nat Neurosci 9, 723-727.
Altman, J., and Dasq, G. D. (1965). Autoradiographic and histological evidence of postnatal hippocampal neurogenesis in rats. J Comp Neurol, 319-336.
Alvarez-Buylla, A., and García-Verdugo, J. M. (2002). Neurogenesis in adult subventricular zone. J Neurosci 22, 629-634.
Alvarez-Buylla, A., and Lim, D. A. (2004). For the long run: Maintaining germinal niches in the adult brain. Neuron 41, 683-686.
Aoki, K., Uchihara, T., Sanjo, N., Nakamura, A., Ikeda, K., Tsuchiya, K., and Wakayama, Y. (2003). Increased expression of neuronal apolipoprotein E in human brain with cerebral infarction. Stroke 34, 875-880.
Bolteus, A. J., and Bordey, A. (2004). GABA release and uptake regulate neuronal precursor migration in the postnatal subventricular zone. J Neurosci 24, 7623-7631.
Bour, A., Grootendorst, J., Vogel, E., Kelche, C., Dodart, J. C., Bales, K., Moreau, P. H., Sullivan, P. M., and Mathis, C. (2008). Middle-aged human apoE4 targeted-replacement mice show retention deficits on a wide range of spatial memory tasks. Behav Brain Res 193, 174-182.
Brecht, W. J., Harris, F. M., Chang, S., Tesseur, I., Yu, G.-Q., Xu, Q., Fish, J. D., Wyss-Coray, T., Buttini, M., Mucke, L., et al. (2004). Neuron-specific apolipoprotein E4 proteolysis is associated with increased tau phosphorylation in brains of transgenic mice. J Neurosci 24, 2527-2534.
Brewer, G. J., and Torricelli, J. R. (2007). Isolation and culture of adult neurons and neurospheres. Nat Protocols 2, 1490-1498.
Cameron, H. A., Woolley, C. S., McEwen, B. S., and Gould, E. (1993). Differentiation of newly born neurons and glia in the dentate gyrus of the adult rat. Neuroscience 56, 337-344.
Carleton, A., Petreanu, L. T., Lansford, R., Alvarez-Buylla, A., and Lledo, P.-M. (2003). Becoming a new neuron in the adult olfactory bulb. Nat Neurosci 6, 507-518.
Caselli, R. J., Dueck, A. C., Osborne, D., Sabbagh, M. N., Connor, D. J., Ahern, G. L., Baxter, L. C., Rapcsak, S. Z., Shi, J., Woodruff, B. K., Locke, D. E. C., Snyder, C. H., Alexander, G. E., Rademakers, R., and Reiman, E. M. (2009). Longitudinal modeling of age-related memory decline and the APOE ε4 effect. N Engl J Med 361, 255-263.
Chang, S., Ma, T. R., Miranda, R. D., Balestra, M. E., Mahley, R. W., and Huang, Y. (2005). Lipid- and receptor-binding regions of apolipoprotein E4 fragments act in concert to cause mitochondrial dysfunction and neurotoxicity. Proc Natl Acad Sci USA 102, 18694-18699.
Chen, J., Zhou, Y., Mueller-Steiner, S., Chen, L.-F., Kwon, H., Yi, S., Mucke, L., and Gan, L. (2005). SIRT1 protects against microglia-dependent Amyloid-® toxicity through inhibiting NF-κB signaling. j Biol Chem 280, 40364-40374.
Christie, B. R., and Cameron, H. A. (2006). Neurogenesis in the adult hippocampus. Hippocampus 16, 199-207.
Cohen, R. M., Small, C., Lalonde, F., Friz, J., and Sunderland, T. (2001). Effect of apolipoprotein E genotype on hippocampal volume loss in aging healthy women. Neurology 57, 2223-2228.
Corder, E. H., Saunders, A. M., Strittmatter, W. J., Schmechel, D. E., Gaskell, P. C., Small, G. W., Roses, A. D., Haines, J. L., and Pericak-Vance, M. A. (1993). Gene dose of apolipoprotein E type 4 allele and the risk of Alzheimer's disease in late onset families. Science 261, 921-923.
Duan, X., Chang, J. H., Ge, S., Faulkner, R. L., Kim, J. Y., Kitabatake, Y., Liu, X.-B., Yang, C.-H., Jordan, J. D., Ma, D. K., et al. (2007). Disrupted-in-schizophrenia 1 regulates integration of newly generated neurons in the adult brain. Cell 130, 1146-1158.
Earnheart, J. C., Schweizer, C., Crestani, F., Iwasato, T., Itohara, S., Mohler, H., and Lüscher, B. (2007). GABAergic control of adult hippocampal neurogenesis in relation to behavior indicative of trait anxiety and depression states. J Neurosci 27, 3845-3854.
Eriksson, P. S., Perfilieva, E., Bjork-Erikkson, T., Alborn, A.-M., Nordborg, C., Peterson, D. A., and Gage, F. H. (1998). Neurogenesis in the adult human hippocampus. Nat Med 4, 1313-1317.
Espósito, M. S., Piatti, V. C., Laplagne, D. A., Morgenstern, N. A., Ferrari, C. C., Pitossi, F. J., and Schinder, A. F. (2005). Neuronal differentiation in the adult hippocampus recapitulates embryonic development. J Neurosci 25, 10074-10086.
Fan, X., Xu, H., Cai, W., Yang, Z., and Zhang, J. (2003). Spatial and temporal patterns of expression of Noggin and BMP4 in embryonic and postnatal rat hippocampus. Developmental Brain Res 146, 51-58.
Galvão, R. P., Garcia-Verdugo, J. M., and Alvarez-Buylla, A. (2008). Brain-derived neurotrophic factor signaling does not stimulate subventricular zone neurogenesis in adult mice and rats. J Neurosci 28, 13368-13383.
Ganguly, K., Schinder, A. F., Wong, S. T., and Poo, M.-M. (2001). GABA itself promotes the developmental switch of neuronal GABAergic responses from excitation to inhibition. Cell 105, 521-532.
Ge, S., Goh, E. L. K., Sailor, K. A., Kitabatake, Y., Ming, G.-L., and Song, H. (2006a). GABA regulates synaptic integration of newly generated neurons in the adult brain. Nature 439, 589-593.
Ge, S., Pradhan, D. A., Ming, G.-L., and Song, H. (2006b). GABA sets the tempo for activity-dependent adult neurogenesis. Trends Neurosci 30, 1-8.
Götz, J., Probst, A., Spillantini, M. G., Schäfer, T., Jakes, R., Bürki, K., and Goedert, M. (1995). Somatodendritic localization and hyperphosphorylation of tau protein in transgenic mice expressing the longest human brain tau isoform. EMBO J. 14, 1304-1313.
Grootendorst, J., Bour, A., Vogel, E., Kelche, C., Sullivan, P. M., Dodart, J.-C., Bales, K., and Mathis, C. (2005). Human apoE targeted replacement mouse lines: h-apoE4 and h-apoE3 mice differ on spatial memory performance and avoidance behavior. Behav Brain Res 159, 1-14.
Harris, F. M., Brecht, W. J., Xu, Q., Tesseur, I., Kekonius, L., Wyss-Coray, T., Fish, J. D., Masliah, E., Hopkins, P. C., Scearce-Levie, K., et al. (2003). Carboxyl-terminal-truncated apolipoprotein E4 causes Alzheimer's disease-like neurodegeneration and behavioral deficits in transgenic mice. Proc Natl Acad Sci USA 100, 10966-10971.
Harris, F. M., Tesseur, I., Brecht, W. J., Xu, Q., Mullendorff, K., Chang, S., Wyss-Coray, T., Mahley, R. W., and Huang, Y. (2004). Astroglial regulation of apolipoprotein E expression in neuronal cells. Implications for Alzheimer's disease. J Biol Chem 279, 3862-3868.
Huang, Y. (2006a). Apolipoprotein E and Alzheimer disease. Neurology 66 (Suppl. 1), S79-S85.
Huang, Y. (2006b). Molecular and cellular mechanisms of apolipoprotein E4 neurotoxicity and potential therapeutic strategies. Curr Opin Drug Discov Dev 9, 627-641.
Huang, Y., Liu, X. Q., Wyss-Coray, T., Brecht, W. J., Sanan, D. A., and Mahley, R. W. (2001). Apolipoprotein E fragments present in Alzheimer's disease brains induce neurofibrillary tangle-like intracellular inclusions in neurons. Proc Natl Acad Sci USA 98, 8838-8843.
Huang, Y., Weisgraber, K. H., Mucke, L., and Mahley, R. W. (2004). Apolipoprotein E. Diversity of cellular origins, structural and biophysical properties, and effects in Alzheimer's disease. J Mol Neurosci 23, 189-204.
Ishihara, T., Hong, M., Zhang, B., Nakagawa, Y., Lee, M. K., Trojanowski, J. Q., and Lee, V. M.-Y. (1999). Age-dependent emergence and progression of a tauopathy in transgenic mice overexpressing the shortest human tau isoform. Neuron 24, 751-762.
Jacobs, S., Lie, D. C., DeCicco, K. L., Shi, Y., DeLuca, L. M., Gage, F. H., and Evans, R. M. (2006). Retinoic acid is required early during adult neurogenesis in the dentate gyrus. Proc Natl Acad Sci USA 103, 3902-3907.
Kaplan, M. S., and Bell, D. H. (1984). Mitotic neuroblasts in the 9-day-old and 11-month-old rodent hippocampus. J Neurosci 4, 1429-1441.
Kempermann, G., Gast, D., Kronenberg, G., Yamaguchi, M., and Gage, F. H. (2003). Early determination and long-term persistence of adult-generated new neurons in the hippocampus of mice. Development 130, 391-399.
Lee, V. M.-Y., Goedert, M., and Trojanowski, J. Q. (2001). Neurodegenerative tauopathies. Annu Rev Neurosci 24, 1121-1159.
Lie, D.-C., Song, H., Colamarino, S. A., Ming, G.-L., and Gage, F. H. (2004). Neurogenesis in the adult brain: New strategies for central nervous system diseases. Annu Rev PharmacolToxicol 44, 399-421.
Lim, D. A., Tramontin, A. D., Trevejo, J. M., Herrera, D. G., Garcia-Verdugo, J. M., and Alvarez-Buylia, A. (2000). Noggin antagonizes BMP signaling to create a niche for adult neurogenesis. Neuron 28, 713-726.
Liu, X., Wang, Q., Haydar, T. F., and Bordey, A. (2005). Nonsynaptic GABA signaling in postnatal subventricular zone controls proliferation of GFAP-expressing progenitors. Nat Neurosci 8, 1179-1187.
Liu, Z., Neff, R. A., and Berg, D. K. (2006). Sequential interplay of nicotinic and GABAergic signaling guides neuronal development. Science 314, 1610-1613.
Mahley, R. W., Weisgraber, K. H., and Huang, Y. (2006). Apolipoprotein E4: A causative factor and therapeutic target in neuropathology, including Alzheimer's disease. Proc Natl Acad Sci USA 103, 5644-5651.
Ming, G.-L., and Song, H. (2005). Adult neurogenesis in the mammalian central nervous system. Annu Rev Neurosci 28, 223-250.
Moffat, S. D., Szekely, C. A., Zonderman, A. B., Kabani, N. J., and Resnick, S. M. (2000). Longitudinal change in hippocampal volume as a function of apolipoprotein E genotype. Neurology 55, 134-136.
Owens, D. F., and Kriegstein, A. R. (2002). Is there more to GABA than synaptic inhibition? Nat Rev Neurosci 3, 715-727.
Raber, J., Wong, D., Buttini, M., Orth, M., Bellosta, S., Pitas, R. E., Mahley, R. W., and Mucke, L. (1998). Isoform-specific effects of human apolipoprotein E on brain function revealed in ApoE knockout mice: Increased susceptibility of females. Proc Natl Acad Sci USA 95, 10914-10919.
Raber, J., Wong, D., Yu, G.-Q., Buttini, M., Mahley, R. W., Pitas, R. E., and Mucke, L. (2000). Apolipoprotein E and cognitive performance. Nature 404, 352-354.
Ray, J., and Gage, F. H. (2006). Differential properties of adult rat and mouse brain-derived neural stem/progenitor cells. Mol Cell Neurosci 31, 560-573.
Rochefort, C., Gheusi, G., Vincent, J.-D., and Liedo, P.-M. (2002). Enriched odor exposure increases the number of newborn neurons in the adult olfactory bulb and improves odor memory. J Neurosci 22, 2679-2689.
Saunders, A. M., Strittmatter, W. J., Schmechel, D., St George-Hyslop, P. H., Pericak-Vance, M. A., Joo, S. H., Rosi, B. L., Gusella, J. F., Crapper-MacLachlan, D. R., Alberts, M. J., et al. (1993). Association of apolipoprotein E allele ε4 with late-onset familial and sporadic Alzheimer's disease. Neurology 43, 1467-1472.
Selkoe, D. J. (1991). The molecular pathology of Alzheimer's disease. Neuron 6, 487-498.
Song, H., Stevens, C. F., and Gage, F. H. (2002). Astroglia induce neurogenesis from adult neural stem cells. Nature 417, 39-44.
Suh, H., Consiglio, A., Ray, J., Sawai, T., D'Amour, K. A., and Gage, F. H. (2007). In vivo fate analysis reveals the multipotent and self-renewal capacities of Sox2+ neural stem cells in the adult hppocampus. Cell Stem Cell 1, 515-528.
Suh, H., Deng, W., and Gage, F. H. (2009). Signaling in Adult Neurogenesis. Annu Rev Cell Dev Biol 25, 11.1-11.23.
Sullivan, P. M., Mezdour, H., Aratani, Y., Knouff, C., Najib, J., Reddick, R. L., Quarfordt, S. H., and Maeda, N. (1997). Targeted replacement of the mouse apolipoprotein E gene with the common human APOE3 allele enhances diet-induced hypercholesterolemia and atherosclerosis. J Biol Chem 272, 17972-17980.
Sullivan, P. M., Mace, B. E., Maeda, N., and Schmechel, D. E. (2004). Marked regional differences of brain human apolipoprotein E expression in targeted replacement mice. Neuroscience 124, 725-733.
Tang, J., Song, M., Wang, Y., Fan, X., Xu, H., and Bai, Y. (2009). Noggin and BMP4 co-modulate adult hippocampal neurogenesis in the APP_{swe}/PSI_{ΔE9} transgenic mouse model of Alzheimer's disease. Biochemical & Biophysical Res Communications 385, 341-345.
Tanzi, R. E., and Bertram, L. (2001). New frontiers in Alzheimer's disease genetics. Neuron 32, 181-184.
Tozuka, Y., Fukuda, S., Namba, T., Seki, T., and Hisatsune, T. (2005). GABAergic excitation promotes neuronal differentiation in adult hippocampal progenitor cells. Neuron 47, 803-815.
van Pragg, H., Schinder, A. F., Christie, B. R., Toni, N., Palmer, T. D., and Gage, F. H. (2002). Functional neurogenesis in the adult hippocampus. Nature 415, 1030-1034.
Villasana, L., Acevedo, S., Poage, C., and Raber, J. (2006). Sex- and APOE isoform-dependent effects of radiation on cognitive function. Radiation Res 166, 883-891.
Xu, P.-T., Gilbert, J. R., Qiu, H.-L., Ervin, J., Rothrock-Christian, T. R., Hulette, C., and Schmechel, D. E. (1999). Specific regional transcription of apolipoprotein E in human brain neurons. Am J Pathol 154, 601-611.
Xu, Q., Bernardo, A., Walker, D., Kanegawa, T., Mahley, R. W., and Huang, Y. (2006). Profile and regulation of apolipoprotein E (apoE) expression in the CNS in mice with targeting of green fluorescent protein gene to the apoE locus. J Neurosci 26, 4985-4994.
Xu, Q., Walker, D., Bernardo, A., Brodbeck, J., Balestra, M. E., and Huang, Y. (2008). Intron-3 retention/splicing controls neuronal expression of apolipoprotein E in the CNS. J Neurosci 28, 1452-1459.
Zhang, C.-L., Zou, Y., He, W., Gage, F. H., and Evans, R. M. (2008). A role for adult TLX-positive neural stem cells in learning and behaviour. Naure 451, 1004-1009.
Zhao, C., Deng, W., and Gage, F. H. (2008). Mechanisms and functional implications of adult neurogenesis. Cell 132, 645-660.
Zhao, C., Teng, E. M., Summers, R. G., Jr., Ming, G.-L., and Gage, F. H. (2006). Distinct morphological stages of dentate granule neuron maturation in the adult mouse hippocampus. J Neurosci 26, 3-11.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of increasing the functionality of a GABAergic interneuron in the hilus of the hippocampus of an individual having at least one apolipoprotein E4 (apoE4) allele, the method comprising administering to the individual an effective amount of an agent that increases GABAergic interneuron function.

2. The method of claim 1, wherein the agent that increases GABAergic function is a gamma-aminobutyric acid-A (GABA_{A}) receptor agonist, a selective inhibitor of gamma-aminobutyric acid (GABA) uptake, an inhibitor of GABA-transaminase, or an agent that stimulates release of GABA from a GABAergic interneuron.

3. The method of claim 1, wherein the agent is a GABA_{A} receptor agonist.

4. The method of claim 3, wherein the GABA_{A} receptor agonist binds at the GABA site.

5. The method of claim 3, wherein the GABA_{A} receptor agonist is a positive allosteric modulator.

6. The method of claim 1, wherein the individual is heterozygous for apoE4.

7. The method of claim 1, wherein the individual is homozygous for apoE4.

8. The method of claim 1, further comprising introducing a neural stem cell (NSC) into the individual.

9. The method of claim 8, wherein the NSC is obtained from a donor individual who is the same as the individual being treated.

10. The method of claim 8, wherein the NSC is obtained from a donor individual who is other than the individual being treated.

11. The method of claim 8, wherein the NSC is an induced NSC (iNSC).

12. The method of claim 11, wherein the iNSC is induced from a somatic cell obtained from the individual being treated.

13. The method of claim 8, wherein the NSC is derived from an induced pluripotent stem cell.

14. The method of claim 8, wherein said introducing results in an increase in the number of newborn mature neurons in the hippocampus of the individual.

15. The method of claim 1, wherein said increase in the functionality of a GABAergic interneuron results in an increase in cognitive function in the individual.

16. The method of claim 15, wherein said cognitive function is memory.

17. The method of claim 15, wherein said cognitive function is learning.


SEQUENCE LISTING (9 sequences)

SEQ ID NO: 1 - protein, 11 aa, Artificial Sequence
  Synthetic peptide
YGRKKRRQRRR

SEQ ID NO: 2 - protein, 12 aa, Artificial Sequence
  Synthetic peptide
RRQRRTSKLMKR

SEQ ID NO: 3 - protein, 27 aa, Artificial Sequence
  Synthetic peptide
GWTLNSAGYLLGKINLKALAALAKKIL

SEQ ID NO: 4 - protein, 33 aa, Artificial Sequence
  Synthetic peptide
KALAWEAKLAKALAKALAKHLAKALAKALKCEA

SEQ ID NO: 5 - protein, 16 aa, Artificial Sequence
  Synthetic peptide
RQIKIWFQNRRMKWKK

SEQ ID NO: 6 - protein, 9 aa, Artificial Sequence
  Synthetic peptide
RKKRRQRRR

SEQ ID NO: 7 - protein, 11 aa, Artificial Sequence
  Synthetic peptide
YARAAARQARA

SEQ ID NO: 8 - protein, 11 aa, Artificial Sequence
  Synthetic peptide
THRLPRRRRRR

SEQ ID NO: 9 - protein, 11 aa, Artificial Sequence
  Synthetic peptide
GGRRARRRRRR